(19) United States
(12) Patent Application Publication
(10) Pub. No.: US 2025/0303180 A1
(43) Pub. Date: Oct. 2, 2025

(54) REQUESTING CHANNEL WAKEUP IN IMPLANTABLE MEDICAL DEVICES

(71) Applicant: Medtronic, Inc., Minneapolis, MN (US)

(72) Inventors: Nicholas Hendrickson, Andover, MN (US); Michael B. Terry, Seattle, WA (US); Evan S. Johnson, New Brighton, MN (US); Jonathan D. Edmonson, Blaine, MN (US)

(21) Appl. No.: 18/866,707

(22) PCT Filed: May 23, 2023
(86) PCT No.: PCT/IB2023/055295

§ 371 Date: Nov. 18, 2024

Related U.S. Application Data
(60) Provisional application No. 63/365,307, filed on May 25, 2022.

(51) Int. Cl. A61N 1/372; A61N 1/365; A61N 1/39

(52) U.S. Cl. CPC A61N 1/37288; A61N 1/36514; A61N 1/39622

(57) ABSTRACT

This disclosure includes example medical device systems, and techniques for communicating between medical devices. An example medical device includes memory configured to store parameters for therapy delivery for a patient, communication circuitry, electrical signal generation circuitry, and processing circuitry. The processing circuitry is configured to control the electrical signal generation circuitry to deliver a first electrical signal to an anatomy of the patient. The processing circuitry is configured to, based on the electrical signal generation circuitry delivering the first electrical signal, control the communication circuitry to communicate with another medical device.

This application claims the benefit of U.S. Provisional Patent Application Ser. No. 63/365,307, filed 25 May 2022, the entire content of which is incorporated herein by reference.

### TECHNICAL FIELD

The disclosure relates to medical devices, and, more particularly, to medical device systems configured to sense physiological parameters of a patient and/or to deliver therapy to the patient.

### BACKGROUND

A wide variety of medical devices for delivering a therapy to or monitoring a physiological condition of a patient have been used clinically or proposed for clinical use in patients. Examples include implantable medical devices (IMDs) that deliver therapy to and/or monitor conditions associated with the heart, muscle, nerve, brain, stomach or other tissue. Some therapies include the delivery of electrical stimulation to such tissues. Some IMDs may employ electrodes for the delivery of therapeutic electrical signals to such organs or tissues, electrodes for sensing intrinsic physiological electrical signals within the patient, which may be propagated by such organs or tissue, and/or other sensors for sensing physiological signals of a patient. Other medical devices may be attached to or otherwise contacting the patient.

In some instances, a patient may have more than one medical device implanted. As an example, implantable cardioverter defibrillators (ICDs) and implantable artificial pacemakers may be implanted to provide cardiac pacing therapy to a patient's heart when the natural pacemaker and/or conduction system of the heart fails to provide synchronized atrial and ventricular contractions at rates and intervals sufficient to sustain healthy patient function. Such anti-bradycardial pacing may provide relief from symptoms, or even life support, for a patient. Cardiac pacing may also provide electrical overdrive stimulation, e.g., anti-tachyarrhythmia pacing (ATP) therapy, to suppress or convert tachyarrhythmias, again supplying relief from symptoms and preventing or terminating arrhythmias that could lead to sudden cardiac death. Additionally, ICDs may deliver anti-tachyarrhythmia shocks in response detecting ventricular tachyarrhythmias, e.g., ventricular tachycardia (VT) or ventricular fibrillation (VF), that is not (or likely will not be) terminated by ATP.

Communication between two or more devices associated with a person, e.g., implanted within the person and/or attached to or otherwise contacting the person, may be desirable in a number of applications, such as for monitoring or managing health of a patient. Communication between these devices may, for example, enable the exchange of information, coordinated monitoring of a health condition and/or coordinated therapy to treat health conditions.

### SUMMARY

Implantable medical devices (IMDs), such as ICDs, cardiac resynchronization therapy (CRT) systems, implantable pulse generators (IPGs), temporary ambulatory pacers (TAPs), and external devices, such as external computing devices, may monitor patient parameters and/or deliver therapy, such as ATP therapy or a shock to the heart of a patient. Such therapy may be delivered to advance the heart to refractory thereby terminating a tachyarrhythmia or to treat myocardial infarction.

Such IMDs may on occasion communicate with one another, for example to provide information collected by a first IMD to a second IMD or to provide instructions to the second IMD, such as to begin delivering therapy, to stop delivering therapy, and/or to configure an operation of the second IMD, such as to reprogram the second IMD. In order to communicate with one another, typically each IMD may monitor for a communication wake-up signal from the other IMD or from an external device on a communication channel. Typically, the amount of data to be transmitted between IMDs is very low, so actually opening the communication channel and transmitting/receiving the data is not power intensive. However, monitoring for the wake-up signal on the communication channel may be relatively power intensive as an IMD may use a relatively large current draw to monitor for the wake-up signal. Because some IMDs generally have a very limited amount of power, it may be desirable to limit, reduce, or remove the monitoring for a communication wake-up signal on a communication channel altogether from at least one of the IMDs. This may reduce power consumption of the IMD, thereby extending battery life and/or time between recharges. As an IMD is implanted within a body, extending battery life may reduce the need for replacement surgery.

The systems, IMDs, and techniques of this disclosure provide for communication between IMDs within a patient without the need for at least one of the IMDs monitoring for a communication wake-up signal on a communication channel. For example, a first IMD may, rather than send a communication wake-up signal, deliver a first electrical signal to an anatomy (e.g., a heart) of a patient. This anatomy may be already monitored by one or more sensors of a second IMD and/or sensing circuitry of the second IMD and therefore, not generally increase current draw. In some examples, the first electrical signal may be configured to generate a physiological response in the anatomy which may be sensed by the one or more sensors of the second IMD and/or by sensing circuitry of the second IMD. In other examples, the first electrical signal may be configured to be directly sensed by the one or more sensors of the second IMD and/or sensing circuitry of the second IMD, which may be actively sensing as a part of other IMD operations. The first electrical signal may be a signal that would not otherwise be delivered by the first IMD (e.g., the purpose of the first electrical signal is not necessarily to provide therapy or monitor a parameter of a patient, such as a signal sent to determine an impedance of target tissue, but is to have the second IMD initiate communication with the first IMD or take some other action). The second IMD may be configured, upon sensing the delivery of the first electrical signal to the anatomy, to communicate with the first IMD. Thus, the first IMD and the second IMD may use non-channel based signaling to request communication therebetween. In this manner, the second IMD does not need to use a communication channel to monitor for a communication wake-up signal, thereby saving power. These techniques may be particularly useful in the case where the second IMD is a very low power device. For example, the second IMD may be a lower power device than the first IMD. While the techniques of this disclosure are generally discussed with regard to two IMDs. Any number of two or more IMDs may implement the techniques discussed herein.

In one example, this disclosure is directed to a medical device including memory configured to store parameters for a first electrical signal for a patient; communication circuitry; electrical signal generation circuitry; and processing circuitry communicatively coupled to the memory, the communication circuitry, and the electrical signal generation circuitry, the processing circuitry being configured to: control the electrical signal generation circuitry to deliver the first electrical signal to an anatomy of the patient; and based on the electrical signal generation circuitry delivering the first electrical signal, control the communication circuitry to communicate with another medical device.

In another example, this disclosure is directed to a medical device including memory configured to store parameters indicative of delivery of a first electrical signal to an anatomy of a patient; communication circuitry; at least one of a sensor or sensing circuitry; and processing circuitry communicatively coupled to the memory, the communication circuitry, and the at least one of the sensor or the sensing circuitry, the processing circuitry being configured to: receive from the at least one of the sensor or the sensing circuitry a signal; determine that the signal is indicative of the delivery of the first electrical signal by another medical device to the anatomy of the patient; based on receiving the signal indicative of the delivery of the first electrical signal, control the communication circuitry to initiate communication with at least one of the another medical device or a third medical device.

In another example, this disclosure is directed to a method including: controlling, by processing circuitry, electrical signal generation circuitry to deliver a first electrical signal to an anatomy of a patient; and controlling, by the processing circuitry and based on the electrical signal generation circuitry delivering the first electrical signal, communication circuitry to communicate with another medical device.

In another example, this disclosure is directed to a method including: receiving, by processing circuitry of a medical device and from at least one of a sensor or sensing circuitry, a signal; determining that the signal is indicative of the delivery of a first electrical signal by another medical device to an anatomy of a patient; controlling, by the processing circuitry and based on receiving the signal indicative of the delivery of the first electrical signal, the communication circuitry to initiate communication with at least one of the another medical device or a third medical device.

In another example, this disclosure is directed to a method including: controlling, by first processing circuitry of a first medical device, electrical signal generation circuitry to deliver a first electrical signal to an anatomy of a patient; receiving, by second processing circuitry of a second medical device and from at least one of a sensor or sensing circuitry, a signal indicative of the delivery of the first electrical signal from the first medical device; and controlling, by the second processing circuitry and based on receiving the signal indicative of the delivery of the first electrical signal, second communication circuitry to initiate communication with at least one of the first medical device or a third medical device.

In another example, this disclosure is directed to a non-transitory computer-readable storage medium having stored thereon instructions that, when executed, cause processing circuitry to: control the electrical signal generation circuitry to deliver a first electrical signal; and based on the electrical signal generation circuitry delivering the first electrical signal, communicate with another medical device via communication circuitry.

In a further example, this disclosure is directed to a non-transitory computer-readable storage medium having stored thereon instructions that, when executed, cause processing circuitry to: receive from at least one of a sensor or sensing circuitry a signal; determine that the signal is indicative of the delivery of a first electrical signal from another medical device to anatomy of a patient; and based on receiving the signal indicative of the delivery of the first electrical signal, control communication circuitry to initiate communication with at least one of the another medical device or a third medical device.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

FIG. 1A is a front view of a patient implanted with an example implantable medical device system.

FIG. 1B is a side view of the patient implanted with the implantable medical device system of FIG. 1A.

FIG. 1C is a transverse view the patient implanted with the implantable medical device system of FIGS. 1A and 1B.

FIG. 2 is a conceptual drawing illustrating an example configuration of the intracardiovascular pacing device (IPD) of the implantable medical device system of FIGS. 1A-1C.

FIG. 3 is a functional block diagram illustrating an example configuration of the IPD of FIGS. 1A-1C and 2.

FIG. 4 is a functional block diagram illustrating an example configuration of the ICD of the implantable medical device system of FIGS. 1A-1C.

FIG. 5 is a functional block diagram illustrating an example configuration of the external device of FIGS. 1A-1C.

FIG. 6 is a flowchart illustrating example communication techniques for a first IMD in accordance with one or more aspects of this disclosure.

FIG. 7 is a flowchart illustrating example communication techniques for a second IMD in accordance with one or more aspects of this disclosure.

FIG. 8 is a flowchart illustrating example communication techniques for a first IMD and a second IMD in accordance with one or more aspects of this disclosure

### DETAILED DESCRIPTION

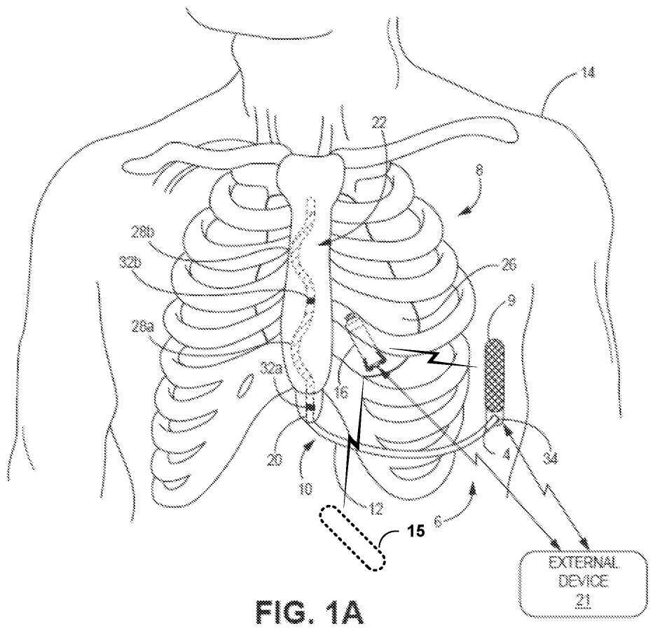

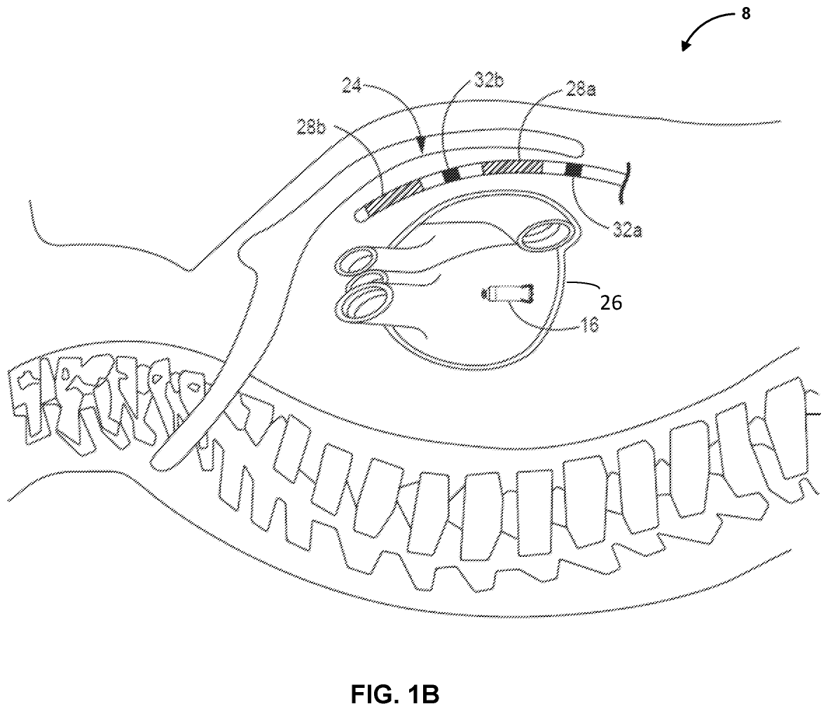

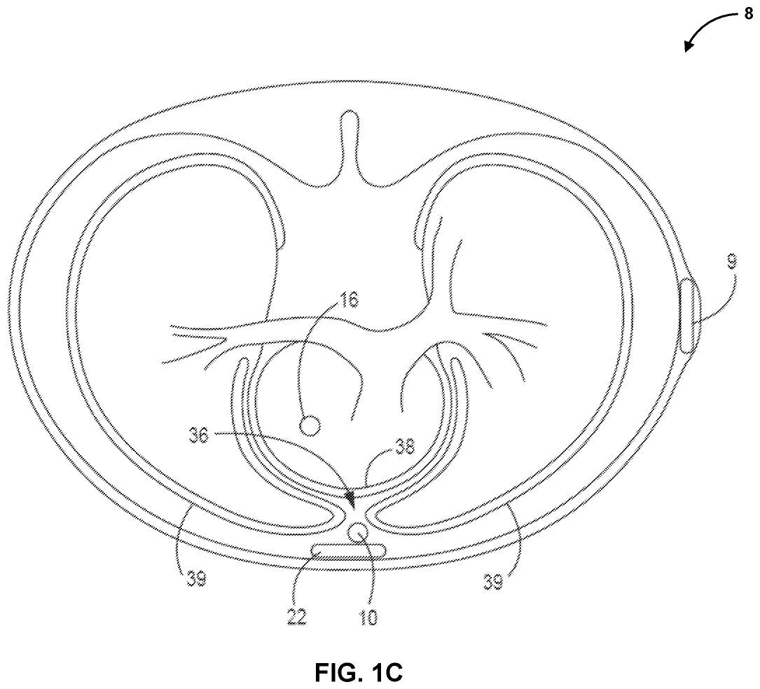

FIGS. 1A-1C are conceptual diagrams illustrating various views of an example implantable medical device system 8. The system 8 includes an extracardiovascular ICD system 6, including ICD 9 connected to a medical electrical lead 10, and IPD 16 configured in accordance with the principles of the present application. FIG. 1A is a front view of a patient 14 implanted with the medical device system 8. FIG. 1B is a side view of patient 14 implanted with the medical device system 8. FIG. 1C is a transverse view of patient 14 implanted with the medical device system 8.

The ICD 9 may include a housing that forms a hermetic seal that protects components of the ICD 9. The housing of the ICD 9 may be formed of a conductive material, such as titanium or titanium alloy, which may function as a housing electrode (sometimes referred to as a can electrode). In other examples, the ICD 9 may be formed to have or may include one or more electrodes on the outermost portion of the housing. The ICD 9 may also include a connector assembly (also referred to as a connector block or header) that includes electrical feedthroughs through which electrical connections are made between conductors of lead 10 and electronic components included within the housing of the ICD 9. As will be described in further detail herein, housing may house one or more processors, memories, transmitters, receivers, sensors, sensing circuitry, electrical signal generation circuitry, power sources and other appropriate components. The housing is configured to be implanted in a patient, such as patient 14.

ICD 9 is implanted extra-thoracically on the left side of patient 14, e.g., under the skin and outside the ribcage (subcutaneously or submuscularly). ICD 9 may, in some instances, be implanted between the left posterior axillary line and the left anterior axillary line of patient 14. ICD 9 may, however, be implanted at other extra-thoracic locations on patient 14 as described later.

Lead 10 may include an elongated lead body 12 sized to be implanted in an extracardiovascular location proximate the heart, e.g., intra-thoracically (as illustrated in FIGS. 1A-C), subcutaneously, or intercostally for example. In the illustrated example, lead body 12 extends superiorly intra-thoracically underneath the sternum, in a direction substantially parallel to the sternum. In one example, the distal portion 24 of lead 10 may reside in a substernal location such distal portion 24 of lead 10 extends superior along the posterior side of the sternum substantially within the anterior mediastinum 36. Anterior mediastinum 36 may be viewed as being bounded laterally by pleurae 39, posteriorly by pericardium 38, and anteriorly by the sternum 22. In some instances, the anterior wall of anterior mediastinum 36 may also be formed by the transversus thoracis and one or more costal cartilages. Anterior mediastinum 36 includes a quantity of loose connective tissue (such as areolar tissue), adipose tissue, some lymph vessels, lymph glands, substernal musculature (e.g., transverse thoracic muscle), the thymus gland, branches of the internal thoracic artery, and the internal thoracic vein. Lead 10 may be implanted at other locations, such as over the sternum, offset to the right of the sternum, angled lateral from the proximal or distal end of the sternum, or the like. In other examples, distal portion of lead 10 may reside within a blood vessel within the substernal location, such as within an internal thoracic artery, an internal thoracic vein, an intercostal vein, the superior epigastric vein, or the azygos, hemiazygos, or accessory hemiazygos veins, as examples. In this manner, lead 10 remains outside the heart in an extra-cardiac location.

Lead body 12 may have a generally tubular or cylindrical shape and may define a diameter of approximately 3-9 French (Fr), however, lead bodies of less than 3 Fr and more than 9 Fr may also be utilized. In another configuration, lead body 12 may have a flat, ribbon, or paddle shape with solid, woven filament, or metal mesh structure, along at least a portion of the length of lead body 12. In such an example, the width across lead body 12 may be between 1-3.5 mm. Other lead body designs may be used without departing from the scope of this application.

Lead body 12 of lead 10 may be formed from a non-conductive material, including silicone, polyurethane, fluoropolymers, mixtures thereof, and other appropriate materials, and shaped to form one or more lumens (not shown), however, the techniques are not limited to such constructions. Distal portion 24 may be fabricated to be biased in a desired configuration, or alternatively, may be manipulated by the user into the desired configuration. For example, distal portion 24 may be composed of a malleable material such that the user can manipulate distal portion 24 into a desired configuration where it remains until manipulated to a different configuration. However, distal portion 24 may take on different configurations, including a straight configuration, circular configuration or any of a number of configurations.

Lead body 12 may include a proximal end 4 and a distal portion 24 configured to deliver electrical energy to the heart or sense electrical energy of the heart. Distal portion 24 may be anchored to a desired position within the patient, for example, substernally or subcutaneously by, for example, suturing distal portion 24 to the patient's musculature, tissue, or bone at the xiphoid process entry site. Alternatively, distal portion 24 may be anchored to the patient or through the use of rigid tines, prongs, barbs, clips, screws, and/or other projecting elements or flanges, disks, pliant tines, flaps, porous structures such as a mesh-like element and metallic or non-metallic scaffolds that facilitate tissue growth for engagement, bio-adhesive surfaces, and/or any other non-piercing elements.

Distal portion 24 includes defibrillation electrode 28 configured to deliver a cardioversion/defibrillation shock to the patient's heart. Defibrillation electrode 28 may include a plurality of sections or segments 28a and 28b spaced a distance apart from each other along the length of distal portion 24. The defibrillation electrode segments 28a and 28b may be a disposed around or within lead body 12 of distal portion 24, or alternatively, may be embedded within the wall of lead body 12. In one configuration, defibrillation electrode segments 28a and 28b may each be a coil electrode formed by a conductor. The conductor may be formed of one or more conductive polymers, ceramics, metal-polymer composites, semiconductors, metals or metal alloys, including but not limited to, one of or a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole and other polymers. In another configuration, each of the defibrillation electrodes segments 28a and 28b may be a flat ribbon electrode, a paddle electrode, a braided or woven electrode, a mesh electrode, a directional electrode, a patch electrode or another type of electrode configured to deliver a cardioversion/defibrillation shock to the patient's heart.

In one configuration, the defibrillation electrode segments 28a and 28b are spaced approximately 0.25-4.5 cm, and in some instances between 1-3 cm apart from each other. In another configuration, the defibrillation electrode segments 28a and 28b are spaced approximately 0.25-1.5 cm apart from each other. In a further configuration, the defibrillation electrode segments 28a and 28b are spaced approximately 1.5-4.5 cm apart from each other. In the configuration shown in FIGS. 1A-1C, the defibrillation electrode segments 28a and 28b span a substantial part of distal portion 24. Each of the defibrillation electrode segments 28a and 28b may be between approximately 1-10 cm in length and, more preferably, between 2-6 cm in length and, even more preferably, between 3-5 cm in length. However, lengths of greater than 10 cm and less than 1 cm may be utilized without departing from the scope of this disclosure. A total length of defibrillation electrode 28 (e.g., length of the two segments 28a and 28b combined) may vary depending on a number of variables. The defibrillation electrode 28 may, in one example, have a total length of between approximately 5-10 cm. However, the defibrillation electrode segments 28a and 28b may have a total length less than 5 cm and greater than 10 cm in other examples. In some instances, defibrillation segments 28a and 28b may be approximately the same length or, alternatively, different lengths.

The defibrillation electrode segments 28a and 28b may be electrically connected to one or more conductors, which may be disposed in the body wall of lead body 12 or may alternatively be disposed in one or more insulated lumens (not shown) defined by lead body 12. In an example configuration, each of the defibrillation electrode segments 28a and 28b is connected to a common conductor such that a voltage may be applied simultaneously to all the defibrillation electrode segments 28a and 28b to deliver a defibrillation shock to a patient's heart. In other configurations, the defibrillation electrode segments 28a and 28b may be attached to separate conductors such that each defibrillation electrode segment 28a or 28b may apply a voltage independent of the other defibrillation electrode segments 28a or 28b. In this case, ICD 9 or lead 10 may include one or more switches or other mechanisms to electrically connect the defibrillation electrode segments together to function as a common polarity electrode such that a voltage may be applied simultaneously to all the defibrillation electrode segments 28a and 28b in addition to being able to independently apply a voltage.

In one example, the distance between the closest defibrillation electrode segment 28a and 28b and electrodes 32a and 32b is greater than or equal to 2 mm and less than or equal to 1.5 cm. In another example, electrodes 32a and 32b may be spaced apart from the closest one of defibrillation electrode segments 28a and 28b by greater than or equal to 5 mm and less than or equal to 1 cm. In a further example, electrodes 32a and 32b may be spaced apart from the closest one of defibrillation electrode segments 28a and 28b by greater than or equal to 6 mm and less than or equal to 8 mm.

Electrodes 32a and 32b may be configured to deliver low-voltage electrical pulses to the heart and/or may sense a cardiac electrical activity, e.g., depolarization and repolarization of the heart. As such, electrodes 32a and 32b may be referred to herein as pace/sense electrodes 32a and 32b. In one configuration, electrodes 32a and 32b are ring electrodes. However, in other configurations the electrodes 32a and 32b may be any of a number of different types of electrodes, including ring electrodes, short coil electrodes, paddle electrodes, hemispherical electrodes, directional electrodes, or the like. Electrodes 32a and 32b may be the same or different types of electrodes. Electrodes 32a and 32b may be electrically isolated from an adjacent defibrillation segment 28a or 28b by including an electrically insulating layer of material between electrodes 32a and 32b and the adjacent defibrillation segments 28a and 28b. Each electrode 32a or 32b may have its own separate conductor such that a voltage may be applied to each electrode independently from another electrode 32a or 32b in distal portion 24. In other configurations, each electrode 32a or 32b may be coupled to a common conductor such that each electrode 32a or 32b may apply a voltage simultaneously.

Proximal end 4 of lead body 12 may include one or more connectors 34 to electrically couple lead 10 to ICD 9 subcutaneously implanted within the patient, for example, under the left armpit of the patient. The ICD 9 may include a housing that forms a hermetic seal which protects the components of ICD 9. The housing of ICD 9 may be formed of a conductive material, such as titanium or titanium alloy, which may function as a housing electrode for a particular therapy vector between the housing and distal portion 24. ICD 9 may also include a connector assembly that includes electrical feedthroughs through which electrical connections are made between the one or more connectors 34 of lead 10 and the electronic components included within the housing. The housing of ICD 9 may house one or more processors, memories, transmitters, receivers, sensors, sensing circuitry, therapy circuitry, power sources (capacitors and batteries) and/or other appropriate components. The components of ICD 9 may generate and deliver electrical therapy such as antitachyarrhythmia shocks, and ATP or other cardiac pacing.

The inclusion of electrodes 32a and 32b adjacent to defibrillation electrode segments 28a and 28b provides a number of therapy vectors for the delivery of electrical stimulation therapy to the heart. For example, as shown in FIGS. 1A-1C, at least a portion of the defibrillation electrode 28 and one of the electrodes 32a and 32b may be disposed over the right ventricle, or any chamber of the heart, such that pacing pulses and defibrillation shocks may be delivered to the heart. The housing of ICD 9 may be charged with or function as a polarity different than the polarity of the one or more defibrillation electrode segments 28a and 28b and/or electrodes 32a and 32b such that electrical energy may be delivered between the housing and the defibrillation electrode segment(s) 28a and 28b and/or electrode(s) 32a and 32b to the heart. Each defibrillation electrode segment 28a or 28b may have the same polarity as every other defibrillation electrode segment 28a or 28b when a voltage is applied to it such that a defibrillation shock may be delivered from the entirety of the defibrillation electrode 28. In examples in which defibrillation electrode segments 28a and 28b are electrically connected to a common conductor within lead body 12, this is the only configuration of defibrillation electrode segments 28a and 28b. However, in other examples, defibrillation electrode segments 28a and 28b may be coupled to separate conductors within lead body 12 and may therefore each have different polarities such that electrical energy may flow between defibrillation electrode segments 28a and 28b (or between one of defibrillation electrode segments 28a and 28b and one or more pace/sense electrodes 32a and 32b or the housing electrode) to provide pacing therapy and/or to sense cardiac depolarizations. In this case, the defibrillation electrode segments 28a and 28b may still be electrically coupled together (e.g., via one or more switches within ICD 9) to have the same polarity to deliver a defibrillation shock from the entirety of the defibrillation electrode 28.

Additionally, each electrode 32a and 32b may be configured to conduct electrical pulses directly to the heart, or sense a cardiac depolarization between adjacent defibrillation electrode segments 28a and 28b, whether disposed on the same defibrillation electrode segment 28a or 28b or on other defibrillation electrode segment 28a or 28b, and/or between proximate electrodes 32a and 32b. Additionally, electrodes 32a and 32b may conduct electrical pulses between one another, e.g., between one of electrodes 32a and 32b and an inferior and superior electrode 32a and 32b, between one of electrodes 32a and 32b and the housing electrode, or between a plurality of electrodes 32a and 32b (at the same polarity) and the housing electrode at the opposite polarity. As such, each electrode 32a or 32b may have the same polarity as every other electrode 32a or 32b or alternatively, may have different polarities such that different therapy vectors can be utilized to deliver pacing pulses to the heart.

Electrodes 32a and 32b may also be configured to deliver a first electrical signal to the heart of patient 14. This first electrical signal may include one or more pacing pulses and may be a signal exclusively used by ICD 9 to inform IPD 16 that IPD 16 should communicate with ICD 9 and/or optionally, a third IMD 15. For example, the first electrical signal may be a sub-threshold signal, a double pulse, or some other type of signal that would normally not be delivered by ICD 9 unless ICD 9 is attempting to initiate communication with IPD 16 (or another IMD not shown in FIG. 1A). In some examples, the first electrical signal may not include pacing pulses, but may include other type(s) of signals. For example, the first electrical signal may include a configuration of a voltage signal or a current signal that may not otherwise be used to determine an impedance of target tissue.

IPD 16 may be implanted within a heart 26 of patient 14. In the example of FIGS. 1A-1C, IPD 16 is implanted within the right ventricle of heart 26 to sense electrical activity of heart 26 and deliver pacing therapy, e.g., ATP therapy, to heart 26. IPD 16 may be attached to an interior wall of the right ventricle of heart 26 via one or more fixation elements that penetrate the tissue. These fixation elements may secure IPD 16 to the cardiac tissue and retain an electrode (e.g., a cathode or an anode) in contact with the cardiac tissue. However, in other examples, system 8 may include additional pacing devices 16 within respective chambers of heart 26 (e.g., right or left atrium and/or left ventricle). In further examples, a cardiac pacing device configured similarly to IPD 16 may be attached to an external surface of heart 26 (e.g., in contact with the epicardium) such that the pacing device is disposed outside of heart 26.

IPD 16 may be capable of sensing electrical signals using the electrodes carried on the housing of IPD 16. These electrical signals may be electrical signals generated by cardiac muscle and indicative of depolarizations and repolarizations of heart 26 at various times during the cardiac cycle. IPD 16 may also be capable of sensing electrical signals originating from ICD 9 that are delivered to heart 26, such as therapy signals or a first electrical signal that is delivered to heart 26 by ICD 9 to inform IPD 16 that IPD 16 should initiate communication with ICD 9. This first electrical signal may be a unique signal configured to inform IPD 16 that IPD 16 should initiate communication with ICD 9. To initiate communication, IPD 16 may communicate with ICD 9, such as transmit one or more pairing advertisements, such as Bluetooth pairing advertisements, to ICD 9, or IPD 16 may begin polling on its communication channel.

For example, the first electrical signal may be a signal that ICD 9 would otherwise not deliver to heart 26. The communication between IPD 16 and ICD 9 may be Bluetooth, tissue conduction communication, or any other communication channel type that may otherwise require channel monitoring for a wake-up signal.

IPD 16 may analyze the sensed electrical signals to detect tachyarrhythmias, such as ventricular tachycardia or ventricular fibrillation, or to detect the first electrical signal being delivered by ICD 9 to heart 26. In response to detecting the tachyarrhythmia, IPD 16 may, e.g., depending on the type of tachyarrhythmia, begin to deliver ATP therapy via the electrodes of IPD 16. In response to detecting the first electrical signal, IPD 16 may initiate communication with ICD 9.

IPD 16 and ICD 9 may be configured to communicate with one another, e.g., via radio-frequency communication, conductive or galvanic communication or other type of communication, to cooperate with one another over a communication channel. For example, IPD 16 and ICD 9 may communicate information, such as sense signals, accelerometer signals, and/or delivered signals, and may coordinate to establish pacing and/or sensing vectors between respective electrodes on ICD 9, IPD 16, and/or lead 10. IPD 16 and ICD 9 may be configured for one-way or two-way communication. In some examples, ICD 9 may be configured to communicate with external device 21, while IPD 16 may not be configured to communicate with external device 21. In such examples, ICD 9 may act as an intermediary between external device 21 and IPD 16. For example, when external device 21 attempts to provide programming to or retrieve sensed information from IPD 16, external device 21 may do so through ICD 9. In this manner, IPD 16 does not need to use a communication channel to monitor for a communication wake-up signal from external device 21.

In some examples, patient 14 may have more than two IMDs, such as having third IMD 15. For example, third IMD 15 may be an insertable cardiac monitors (ICM), a cardiac resynchronization therapy (CRT) system, a temporary ambulatory pacer (TAPs), a neurostimulator, a cardiac assist device, etc. Third IMD 15, IPD 16, and ICD 9 may be configured to communicate with one another, e.g., via radio-frequency communication, conductive or galvanic communication or other type of communication, to cooperate with one another over a communication channel. For example, third IMD 15, IPD 16 and ICD 9 may communicate information, such as sense signals, accelerometer signals, and/or delivered signals, and may coordinate to establish pacing and/or sensing vectors between respective electrodes on ICD 9, IPD 16, third IMD 15 and/or lead 10. Third IMD 15, IPD 16 and ICD 9 may be configured for one-way or two-way communication.

In some examples, external device 21 comprises a handheld computing device, wearable device, tablet, laptop computer, computer workstation, networked computing device, or one or more servers. External device 21 may include a user interface that receives input from a user and provides information to the user, such as the indications and alerts discussed herein. In other examples, the user may also interact with external device 21 remotely via a networked computing device. The user may interact with external device 21 to communicate with ICD 9 and/or IPD 16. For example, the user may interact with external device 21 to send an interrogation request and retrieve therapy delivery data, update therapy parameters that define therapy, manage communication between ICD 9 and/or IPD 16, or perform any other activities with respect to ICD 9 and/or IPD 16. In some examples, the user is a clinician, such as a physician, technician, surgeon, electrophysiologist, or other healthcare professional. In some examples, the user may be patient 14, a family member of patient 14, a friend of patient 14, a caregiver of patient 14, or the like.

External device 21 may also allow the user to define how ICD 9 and/or IPD 16 senses electrical signals (e.g., ECGs), detects arrhythmias such as tachyarrhythmias, delivers therapy, and communicates with other devices of system 8. For example, external device 21 may be used to change tachyarrhythmia detection parameters. In another example, external device 21 may be used to manage therapy parameters that define therapies such as ATP therapy. Moreover, external device 21 may be used to alter communication protocols between ICD 9 and IPD 16. For example, external device 21 may instruct ICD 9 and/or IPD 16 to switch between one-way and two-way communication and/or change which of ICD 9 and/or IPD 16 are tasked with initial detection of arrhythmias.

External device 21 may communicate with ICD 9 and/or IPD 16 via wireless communication using any techniques known in the art. Examples of communication techniques may include, for example, proprietary and non-proprietary radiofrequency (RF) telemetry, but other techniques are also contemplated. In some examples, external device 21 may include a programming head that may be placed proximate to patient 14's body near the ICD 9 and/or IPD 16 implant site in order to improve the quality or security of communication between ICD 9 and/or IPD 16 and external device 21.

In some examples, ICD 9 and IPD 16 may engage in communication with each other and/or with external device 21 to facilitate the appropriate detection of cardiac events, delivery of cardiac therapy, such as anti-tachycardia therapy, and generation/transmission of indications regarding the cardiac event. Anti-tachycardia therapy may include ATP. The communication may include one-way communication in which one device is configured to transmit communication messages and the other device is configured to receive those messages. The communication may instead include two-way communication in which each device is configured to transmit and receive communication messages. Although the examples below describe detection of tachyarrhythmias and the delivery of ATP, ICD 9 and IPD 16 may be configured to communicate with each other and provide for the detection of other cardiac events, e.g., a pause in heartbeat, myocardial infarction, etc., and deliver alternative electrical stimulation therapies. In addition, ICD 9 and IPD 16 may be configured to communicate with each other and provide information regarding diagnostics, device status, or other information from the devices.

The leads and systems described herein may be used at least partially within the substernal space, e.g., within anterior mediastinum of patient, to provide a medical device system. An implanter (e.g., physician) may implant the distal portion of the lead intrathoracically using any of a number of implant tools, e.g., tunneling rod, sheath, or other tool that can traverse the diagrammatic attachments and form a tunnel in the substernal location. For example, the implanter may create an incision near the center of the torso of the patient, e.g., and introduce the implant tool into the substernal location via the incision. The implant tool is advanced from the incision superior along the posterior of the sternum in the substernal location. The distal end of lead 10 is introduced into tunnel via implant tool (e.g., via a sheath). As the distal end of lead 10 is advanced through the substernal tunnel, the distal end of lead 10 is relatively straight. The preformed or shaped undulating portion is flexible enough to be straightened out while routing the lead 10 through a sheath or other lumen or channel of the implant tool. Once the distal end of lead 10 is in place, the implant tool is withdrawn toward the incision and removed from the body of the patient while leaving lead 10 in place along the substernal path. As the implant tool is withdrawn, the distal end of lead 10 takes on its pre-formed undulating configuration. Thus, as the implant tool is withdrawn, the undulating configuration pushes electrodes 32a and 32b toward the left side of sternum compared to electrode segments 28a and 28b. As mentioned above, the implanter may align the electrodes 32a and 32b along the anterior median line (or midsternal line) or the left sternal lines (or left lateral sternal line).

While the techniques of this disclosure may be discussed with respect to system 8 as a whole or with respect to specific devices of system 8, in some examples, the techniques of this disclosure may be performed by other devices, such as ICMs, CRT systems, TAPs, neurostimulators, cardiac assist devices, etc. For example, processing circuitry of one or more devices may cooperate to perform any of the techniques of this disclosure.

Although FIGS. 1A-1C are described in the context of an ICD 9 connected to lead 10 and IPD 16, the techniques may be applicable to other systems. For example, instead of or in addition to ICD 9, a system may include a medical device that includes a lead having a distal portion that is implanted above the sternum (or other extra-thoracic, subcutaneous location or intercostal location) instead of being implanted below the ribs and/or sternum. As another example, instead of an intracardiac pacing device, a pacing system may be implanted having a subcutaneous or submuscular pacemaker and one or more leads connected to and extending from the pacemaker into one or more chambers of the heart or attached to the outside of the heart to provide pacing therapy to the one or more chambers. As another example, a system may have one or more insertable cardiac monitors, pressure sensing devices, neurostimulators, or other IMDs instead of or in addition to an ICD or IPD. As such, the example of FIGS. 1A-1C is illustrated for exemplary purposes only and should not be considered limiting of the techniques described herein.

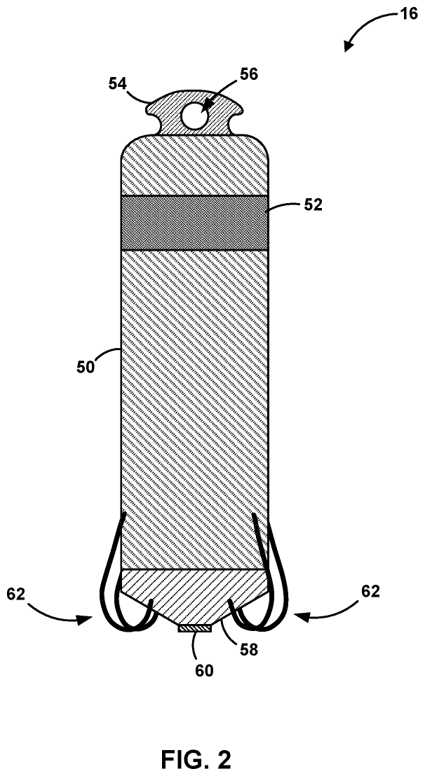

FIG. 2 is a conceptual drawing illustrating an example configuration of IPD 16 of the medical device system of FIGS. 1A-1C. As shown in FIG. 2, IPD 16 includes case 50, cap 58, electrode 60, electrode 52, fixation mechanisms 62, flange 54, and opening 56. Together, case 50 and cap 58 may be considered the housing of IPD 16. In this manner, case 50 and cap 58 may enclose and protect the various electrical components within IPD 16. Case 50 may enclose substantially all of the electrical components, and in some examples, one or more accelerometers, and cap 58 may seal case 50 and create the hermetically sealed housing of IPD 16. Although IPD 16 is generally described as including one or more electrodes, IPD 16 may typically include at least two electrodes (e.g., electrodes 52 and 60) to deliver an electrical signal (e.g., therapy such as ATP) and/or provide at least one sensing vector.

Electrodes 52 and 60 are carried on the housing created by case 50 and cap 58. In this manner, electrodes 52 and 60 may be considered leadless electrodes. In the example of FIG. 2, electrode 60 is disposed on the exterior surface of cap 58. Electrode 60 may be a circular electrode positioned to contact cardiac tissue upon implantation. Electrode 52 may be a ring or cylindrical electrode disposed on the exterior surface of case 50. Both case 50 and cap 58 may be electrically insulating. Electrode 60 may be used as a cathode and electrode 52 may be used as an anode, or vice versa, for delivering pacing stimulation therapy such as ATP. However, electrodes 52 and 60 may be used in any stimulation configuration. In addition, electrodes 52 and 60 may be used to detect intrinsic electrical signals from cardiac muscle and a first electrical signal originating from ICD 9 and being delivered to heart 26 (FIGS. 1A-1C) which is used to inform IPD 16 to initiate communication with ICD 9. In other examples, IPD 16 may include three or more electrodes, where each electrode may deliver therapy and/or detect intrinsic signals and the first electrical signal. ATP delivered by IPD 16, as compared with alternative devices, may be considered to be “painless” to patient 14 or even undetectable by patient 14 since the electrical stimulation occurs very close to or at cardiac muscle and at relatively low energy levels.

Fixation mechanisms 62 may attach IPD 16 to cardiac tissue. Fixation mechanisms 62 may be active fixation tines, screws, clamps, adhesive members, or any other types of attaching a device to tissue. As shown in the example of FIG. 2, fixation mechanisms 62 may be constructed of a memory material that retains a preformed shape. During implantation, fixation mechanisms 62 may be flexed forward to pierce tissue and allowed to flex back towards case 50. In this manner, fixation mechanisms 62 may be embedded within the target tissue.

Flange 54 may be provided on one end of case 50 to enable tethering or extraction of IPD 16. For example, a suture or other device may be inserted around flange 54 and/or through opening 56 and attached to tissue. In this manner, flange 54 may provide a secondary attachment structure to tether or retain IPD 16 within heart 18 if fixation mechanisms 62 fail. Flange 54 and/or opening 56 may also be used to extract IPD 16 once the IPD needs to be explanted (or removed) from patient 14 if such action is deemed necessary.

The techniques described herein may generally be described with regard to a leadless pacing device such as IPD 16. IPD 16 may be an example of an anti-tachycardia pacing device (ATPD). However, alternative implantable medical devices may be used to perform the same or similar functions as IPD 16, e.g., delivering ATP or other therapy to heart 26 and communicate with ICD 9 and/or external device 21.

In another example, the ATPD may be configured to be implanted external to heart 26, e.g., near or attached to the epicardium of heart 26. An electrode carried by the housing of the ATPD may be placed in contact with the epicardium and/or one or more electrodes of leads coupled to the ATPD may be placed in contact with the epicardium at locations sufficient to provide therapy such as ATP (e.g., on external surfaces of the left and/or right ventricles). In any example, subcutaneous ICD 9 may communicate with one or more leadless or leaded devices implanted internal or external to heart 26.

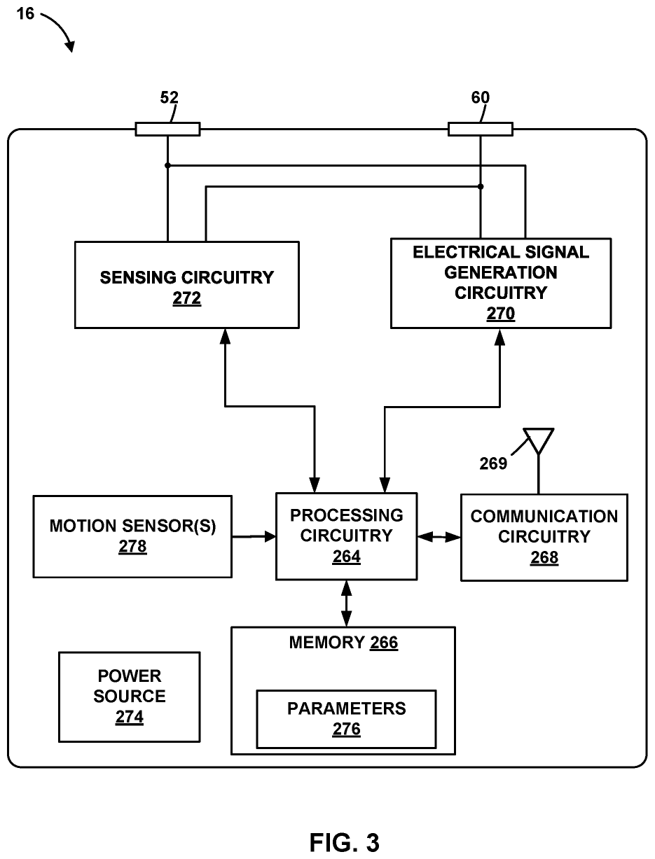

FIG. 3 is a functional block diagram illustrating an example configuration of IPD 16 of FIGS. 1A-1C and FIG. 2. In the illustrated example, IPD 16 includes processing circuitry 264, memory 226, electrical signal generation circuitry 270, sensing circuitry 272, communication circuitry 268, motion sensor(s) 278 (which may include one or more accelerometers), and power source 274. The electronic components may receive power from a power source 274, which may be a rechargeable or non-rechargeable battery. In other examples, IPD 16 may include more or fewer electronic components. The described circuitry may be implemented together on a common hardware component or separately as discrete but interoperable hardware or software components. Depiction of different features as circuitry is intended to highlight different functional aspects and does not necessarily imply that such circuitry must be realized by separate hardware or software components. Rather, functionality associated with one or more circuitries may be performed by separate hardware or software components, or integrated within common or separate hardware or software components.

Memory 226 includes computer-readable instructions that, when executed by processing circuitry 264, cause IPD 16 and processing circuitry 264 to perform various functions attributed to IPD 16 and processing circuitry 264 herein (e.g., determining a cardiac event, determining a fall, a slumping posture, or a change from an upright posture to a non-upright posture, delivering therapy, and/or generating an indication for output). Memory 226 may include any volatile, non-volatile, magnetic, optical, or electrical media, such as a random access memory (RAM), ferroelectric RAM (FRAM), read-only memory (ROM), non-volatile RAM (NVRAM), electrically-erasable programmable ROM (EEPROM), flash memory, or any other digital or analog media.

Processing circuitry 264 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 264 may include multiple components, such as any combination of one or more microprocessors, one or more controllers, one or more DSPs, one or more ASICs, or one or more FPGAs, as well as other discrete or integrated logic circuitry. The functions attributed to processing circuitry 264 herein may be embodied as software, firmware, hardware or any combination thereof.

Electrical signal generation circuitry 270 is electrically coupled to electrodes 52 and 60 carried on the housing of IPD 16. Electrical signal generation circuitry 270 may include one or more pulse generators, capacitors, and/or other components capable of generating and/or storing energy to deliver as pacing therapy. In the illustrated example, electrical signal generation circuitry 270 is configured to generate and deliver electrical stimulation therapy to heart 26. For example, electrical signal generation circuitry 270 may deliver the electrical stimulation therapy to a portion of cardiac muscle within heart 26 via electrodes 52 and 60. In some examples, electrical signal generation circuitry 270 may deliver pacing stimulation, e.g., ATP therapy, in the form of voltage or current electrical pulses. In other examples, electrical signal generation circuitry 270 may deliver stimulation in the form of other signals, such as sine waves, square waves, or other substantially continuous time signals.

Motion sensor(s) 278 may include a three-axis accelerometer, one or more single-axis accelerometers, or the like. Motion sensor(s) 278 may be configured to generate one or more signals that may be indicative of a posture or activity of patient 14.

Processing circuitry 264 may monitor via sensing circuitry 272 signals from heart 26. For example, processing circuitry 264 may monitor heart 26 for electrical signals, including a first electrical signal originating from ICD 9 being delivered to heart 26 which may be indicative of ICD 9 wanting to initiate communication with IPD 16. In response to detecting the delivery of the first electrical signal from ICD 9 to heart 26, processing circuitry 264 may control communication circuitry 268 to initiate communication with ICD 9. In this manner, IPD 16 may monitor for the communication wake-up signal the same sensing circuitry that is already powered to monitor physiological signals, eliminating the need to power the communication circuitry to monitor for the wake-up signal on a separate communication channel.

In some examples, IPD 16 may include a safe mode which processing circuitry 264 may enter to avoid attempting to initiate communication with ICD 9. For example, the safe mode may be desirable to be used when IPD 16 is likely to sense signals similar to the first electrical signal when ICD 9 may not be delivering the first electrical signal to heart 26, such as when patient 14 may be within a magnetic resonance imaging (MRI) machine.

Processing circuitry 264 may control electrical signal generation circuitry 270 to deliver cardiac pacing therapy or other therapy to heart 26 according to parameters, which may be stored in parameters 276. For example, processing circuitry 264 may control electrical signal generation circuitry 270 to deliver pacing pulses with the amplitudes, pulse widths, frequency, or electrode polarities specified by the therapy parameters, including intervals that define under what conditions and when pacing pulses should be delivered. In this manner, electrical signal generation circuitry 270 may deliver pacing pulses (e.g., ATP pulses) to heart 26 via electrodes 52 and 60. Although IPD 16 may only include two electrodes, e.g., electrodes 52 and 60, IPD 16 may utilize three or more electrodes in other examples. IPD 16 may use any combination of electrodes to deliver therapy and/or detect electrical signals from patient 14.

Processing circuitry 264 may control electrical signal generation circuitry 270 to deliver pacing pulses for ATP therapy according to ATP parameters in parameters 276 stored in memory 266. ATP therapy parameters may include pulse intervals, pulse width, current and/or voltage amplitudes, and durations for each pacing mode. In some examples, ATP parameters may include a plurality of ranges of propagation times and associated numbers of pulses of an ATP train, a formula that may be applied to a first propagation time, a linear equation that may be applied to a first propagation time, a continuous function that may be applied to a first propagation time, or a look-up table that may include first propagation times and associated number of pulses of an ATP train. For example, the pulse interval may be based on a fraction of the detected ventricular tachycardia cycle length and be between approximately 150 milliseconds and 500 milliseconds (e.g., between approximately 2.0 hertz and 7.0 hertz), and the pulse width may be between approximately 0.5 milliseconds and 2.0 milliseconds. The amplitude of each pacing pulse may be between approximately 2.0 volts and 10.0 volts. In some examples, the pulse amplitude may be approximately 6.0 V and the pulse width may be approximately 1.5 milliseconds; another example may include pulse amplitudes of approximately 5.0 V and pulse widths of approximately 1.0 milliseconds.

Each train of pulses during ATP may last for a duration of between approximately 70 milliseconds to approximately 15 seconds or be defined as a specific number of pulses.

Processing circuitry 264 controls electrical signal generation circuitry 270 to generate and deliver pacing pulses with any of a number of shapes, amplitudes, pulse widths, or other characteristic to capture the heart. For example, the pacing pulses may be monophasic, biphasic, or multi-phasic (e.g., more than two phases). The pacing thresholds of the heart when delivering pacing pulses may depend upon a number of factors, including location, type, size, orientation, and/or spacing of IPD 16 and/or electrodes 52 and/or 60, physical abnormalities of the heart (e.g., pericardial adhesions or myocardial infarctions), or other factor(s).

In examples in which IPD 16 includes more than two electrodes, electrical signal generation circuitry 270 may include a switch and processing circuitry 264 may use the switch to select, e.g., via a data/address bus, which of the available electrodes are used to deliver pacing pulses. The switch may include a switch array, switch matrix, multiplexer, or any other type of switching device suitable to selectively couple stimulation energy to selected electrodes.

Sensing circuitry 272 is electrically connected to and monitors signals from some or all of electrodes 52 and 60 in order to monitor electrical activity of heart 26, impedance, or other electrical phenomenon, including monitoring for the first electrical signal originating from ICD 9. Sensing may be done to determine heart rates or heart rate variability, or to detect arrhythmias (e.g., tachyarrhythmias or bradycardia) or other electrical signals, such as the first electrical signal. Sensing circuitry 272 may also include a switch to select which of the available electrodes (or electrode polarity) are used to sense the heart activity, depending upon which electrode combination, or electrode vector, is used in the current sensing configuration. In examples with several electrodes, processing circuitry 264 may select the electrodes that function as sense electrodes, i.e., select the sensing configuration, via the switch circuitry within sensing circuitry 272. Sensing circuitry 272 may include one or more detection channels, each of which may be coupled to a selected electrode configuration for detection of cardiac signals via that electrode configuration. Some detection channels may be configured to detect cardiac events, such as fiducial points, QRS complexes, P- or R-waves, and provide indications of the occurrences of such events to processing circuitry 264. Processing circuitry 264 may control the functionality of sensing circuitry 272 by providing signals via a data/address bus.

The components of sensing circuitry 272 may be analog components, digital components or a combination thereof. Sensing circuitry 272 may, for example, include one or more sense amplifiers, filters, rectifiers, threshold detectors, analog-to-digital converters (ADCs) or the like. Sensing circuitry 272 may convert the sensed signals to digital form and provide the digital signals to processing circuitry 264 for processing or analysis. For example, sensing circuitry 272 may amplify signals from the sensing electrodes and convert the amplified signals to multi-bit digital signals by an ADC. Sensing circuitry 272 may also compare processed signals to a threshold to detect the existence of atrial or ventricular depolarizations (e.g., P- or R-waves) and indicate the existence of the atrial depolarization (e.g., P-waves) or ventricular depolarizations (e.g., R-waves) to processing circuitry 264.

Sensing circuitry 272 and/or processing circuitry 264 may also include circuitry for measuring the capture threshold for the delivery of pacing pulses via electrodes 52 and 60. The capture threshold may indicate the voltage and pulse width necessary to induce depolarization of the surrounding cardiac muscle. For example, processing circuitry 264 may periodically control electrical signal generation circuitry 270 to modify the amplitude of pacing pulses delivered to patient 14, and sensing circuitry 272 and/or processing circuitry 264 may detect whether the surrounding cardiac tissue depolarized in response to the pacing pulses, i.e., detected whether there was an evoked response to the pacing pulse. Processing circuitry 264 may determine the capture threshold based on the amplitude where loss of capture occurred.

Processing circuitry 264 may process the signals from sensing circuitry 272 to monitor electrical activity of the heart of the patient and to monitor for the first electrical signal. For example, processing circuitry may monitor for a signal sensed by sensing circuitry 272 that ICD 9 may deliver to an anatomy of patient 14 that may be different than normal therapy which may be delivered by ICD 9, such as a sub-threshold signal, a double pulse, or the like. Processing circuitry 264 may store signals obtained by sensing circuitry 272 as well as any generated ECG waveforms, marker channel data or other data derived based on the sensed signals in memory 266. Processing circuitry 264 may analyze the ECG waveforms and/or marker channel data to detect cardiac events (e.g., tachycardia). In response to detecting the cardiac event, processing circuitry 264 may control electrical signal generation circuitry 270 to deliver the desired therapy to treat the cardiac event, e.g., ATP therapy.

In examples in which IPD 16 includes more than two electrodes, electrical signal generation circuitry 270 may include a switch and processing circuitry 264 may use the switch to select, e.g., via a data/address bus, which of the available electrodes are used to deliver pacing pulses. The switch may include a switch array, switch matrix, multiplexer, or any other type of switching device suitable to selectively couple stimulation energy to selected electrodes. Processing circuitry 264 may select the electrodes to function as signal electrodes, or the signal vector, via the switch circuitry within electrical signal generation circuitry 270. In some instances, the same switch circuitry may be used by both electrical signal generation circuitry 270 and sensing circuitry 272. In other instances, each of sensing circuitry 272 and electrical signal generation circuitry 270 may have separate switch circuitry.

Processing circuitry 264 may include a timing and control circuitry, which may be embodied as hardware, firmware, software, or any combination thereof. The timing and control circuitry may comprise a dedicated hardware circuit, such as an ASIC, separate from other processing circuitry 264 components, such as a microprocessor, or a software module executed by a component of processing circuitry 264, which may be a microprocessor or ASIC. The timing and control circuitry may implement programmable counters. If IPD 16 is configured to generate and deliver pacing pulses to heart 26, such counters may control the basic time intervals associated with DDD, VVI, DVI, VDD, AAI, DDI, DDDR, VVIR, DVIR, VDDR, AAIR, DDIR and other modes of pacing.

Intervals defined by the timing and control circuitry within processing circuitry 264 may include atrial and ventricular pacing escape intervals, refractory periods during which sensed P-waves and R-waves are ineffective to restart timing of the escape intervals, and the pulse widths of the pacing pulses. As another example, the timing and control circuitry may withhold sensing from one or more channels of sensing circuitry 272 for a time interval during and after delivery of electrical stimulation to heart 26. The durations of these intervals may be determined by processing circuitry 264 in response to stored data in memory 226. The timing and control circuitry of processing circuitry 264 may also determine the amplitude of the cardiac pacing pulses.

Interval counters implemented by the timing and control circuitry of processing circuitry 264 may be reset upon sensing of R-waves and P-waves with detection channels of sensing circuitry 272. In examples in which IPD 16 provides pacing, electrical signal generation circuitry 270 may include pacer output circuits that are coupled to electrodes 52 and 60, for example, appropriate for delivery of a bipolar or unipolar pacing pulse to one of the chambers of heart 26. In such examples, processing circuitry 264 may reset the interval counters upon the generation of pacing pulses by electrical signal generation circuitry 270, and thereby control the basic timing of cardiac pacing functions, including ATP or post-shock pacing.

The value of the count present in the interval counters when reset by sensed R-waves and P-waves may be used by processing circuitry 264 to measure the durations of R-R intervals, P-P intervals, P-R intervals and R-P intervals, which are measurements that may be stored in memory 266. Processing circuitry 264 may use the count in the interval counters to detect a tachyarrhythmia event, such as atrial fibrillation, atrial tachycardia, ventricular fibrillation, or ventricular tachycardia. These intervals may also be used to detect the overall heart rate, ventricular contraction rate, and heart rate variability. A portion of memory 266 may be configured as a plurality of recirculating buffers, capable of holding series of measured intervals, which may be analyzed by processing circuitry 264 in response to the occurrence of a pace or sense interrupt to determine whether the patient's heart 26 is presently exhibiting atrial or ventricular tachyarrhythmia. In some examples, an arrhythmia detection method may include any suitable tachyarrhythmia detection algorithms.

In some examples, processing circuitry 264 may determine that tachyarrhythmia has occurred by identification of shortened R-R (or P-P) interval lengths. Generally, processing circuitry 264 detects tachycardia when the interval length falls below 220 milliseconds and fibrillation when the interval length falls below 180 milliseconds. In other examples, processing circuitry 264 may detect ventricular tachycardia when the interval length falls between 330 milliseconds and ventricular fibrillation when the interval length falls below 240 milliseconds. These interval lengths are merely examples, and a user may define the interval lengths as desired, which may then be stored within memory 266. This interval length may need to be detected for a certain number of consecutive cycles, for a certain percentage of cycles within a running window, or a running average for a certain number of cardiac cycles, as examples. In other examples, additional physiological parameters may be used to detect an arrhythmia. For example, processing circuitry 264 may analyze one or more morphology measurements, impedances, or any other physiological measurements to determine that patient 14 is experiencing a tachyarrhythmia.

In the event that an ATP regimen is desired, timing intervals for controlling the generation of ATP therapies by therapy deliver circuitry 270 may be loaded by processing circuitry 264 into the timing and control circuitry based on ATP parameters 276 to control the operation of the escape interval counters therein and to define refractory periods during which detection of R-waves and P-waves is ineffective to restart the escape interval counters for the ATP. An ATP regimen may be desired if processing circuitry 264 detects an atrial or ventricular tachyarrhythmia based on signals from sensing circuitry 272, and/or receives a command from another device or system, such as ICD 9, as examples.

In addition to detecting and identifying specific types of cardiac rhythms and the first electrical signal, sensing circuitry 272 may also sample the detected intrinsic signals to generate an ECG or other time-based indication of cardiac events. Processing circuitry 264 may also be able to coordinate the delivery of pacing pulses from different IPDs implanted in different chambers of heart 26, such as an IPD implanted in atrium and/or an IPD implanted in left ventricle. For example, processing circuitry 264 may identify delivered pulses from other IPDs via sensing circuitry 272 and update pulse timing to accomplish a selected pacing regimen. This detection may be on a pulse-to-pulse or beat-to-beat basis, or on a less frequent basis to make slight modifications to pulse rate over time. In other examples, IPDs may communicate with each other via communication circuitry 268 and/or instructions over a carrier wave (such as a stimulation waveform). In this manner, ATP pacing may be coordinated from multiple IPDs.

IPD 16 may deliver ATP therapy using electrodes 52 and 60 and electrical signal generation circuitry 270 and may sense a local evoked response using the electrodes 52 and 60 and sensing circuitry 272 to sense at a location that is at or near the location of the delivery of the ATP therapy. Another device, such as ICD 9 may sense a global evoked response to the ATP pacing delivered by IPD 16 by sensing at a location that is a substantial distance from the location of the delivery of the ATP therapy. In other examples, the same device, using different electrode vectors, may be used to sense both local and global evoked responses to delivered ATP therapy delivered by the device. The evoked responses may be detected via the hardware of sensing circuitry 272 similar to R-wave, e.g., using a sense amplifier to detect amplitude above a threshold shortly after delivery of a pacing pulse, and/or may be by detected by processing circuitry 264 determining a spike by signal processing a digitized version of ECG signals from electrodes 52 and 60.

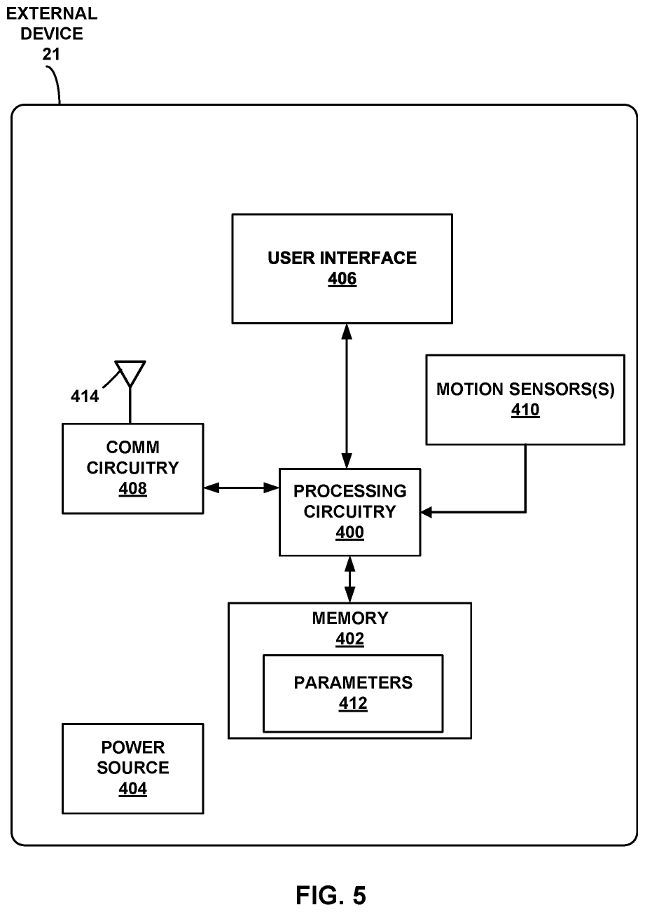

Memory 266 may be configured to store a variety of operational parameters, therapy parameters, including ATP therapy parameters, sensed and detected data, and any other information related to the therapy and treatment of patient 14. In the example of FIG. 5, memory 266 may store sensed ECGs, detected arrhythmias, communications from ICD 9 and/or external device 21, and therapy parameters that define ATP therapy (which may be stored in parameters 276). In other examples, memory 266 may act as a temporary buffer for storing data until it can be uploaded to ICD 9, another implanted device, or external device 21.

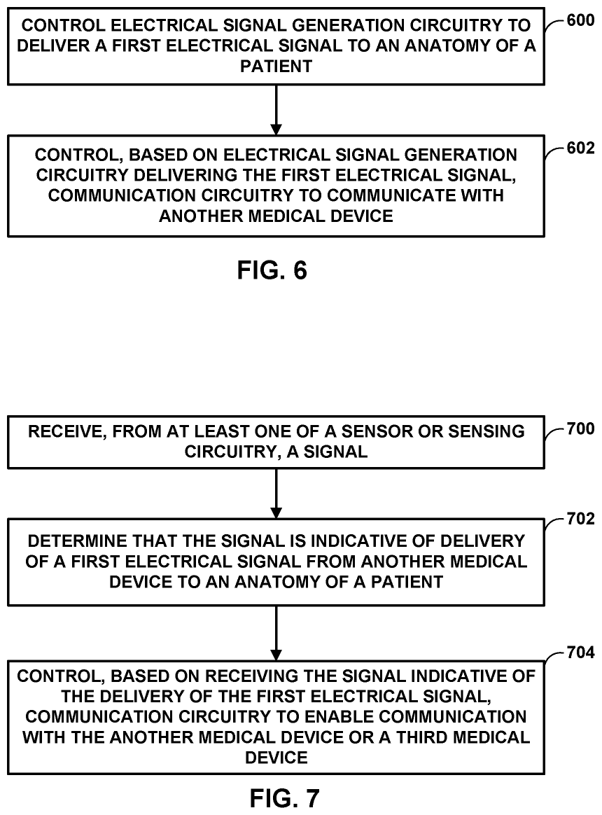

Communication circuitry 268 includes any suitable hardware, firmware, software or any combination thereof for communicating with another device, such as external device 21 (FIGS. 1A-1C and 7), ICD 9 (FIGS. 1A-1C and 6), a clinician programmer, a patient monitoring device, or the like. For example, communication circuitry 284 may include appropriate modulation, demodulation, frequency conversion, filtering, and amplifier components for transmission and reception of data with the aid of antenna 269. Antenna 269 may be located within connector block of IPD 16 or within the housing of IPD 16. Under the control of processing circuitry 264, communication circuitry 268 may receive downlink telemetry from and send uplink telemetry to external device 21 and/or ICD 9 with the aid of antenna 269, which may be internal and/or external. Processing circuitry 264 may provide the data to be uplinked to external device 21 and/or ICD 9 and the control signals for the telemetry circuit within communication circuitry 268, e.g., via an address/data bus. In some examples, communication circuitry 268 may provide received data to processing circuitry 264 via a multiplexer.

In some examples, IPD 16 may signal external device 21 and/or ICD 9 to further communicate with and pass the alert through a network such as the Medtronic CareLink® Network developed by Medtronic plc., of Dublin, Ireland, or some other network linking patient 14 to a clinician. IPD 16 may spontaneously transmit information to the network or in response to an interrogation request from a user.

Power source 274 may be any type of device that is configured to hold a charge to operate the circuitry of IPD 16. Power source 274 may be provided as a rechargeable or non-rechargeable battery. In other example, power source 274 may incorporate an energy scavenging system that stores electrical energy from movement of IPD 16 within patient 14.

The various circuitry of IPD 16 may include any one or more processors, controllers, digital signal processors (DSPs), application specific integrated circuits (ASICs), field programmable gate arrays (FPGAs), or equivalent discrete or integrated circuitry, including analog circuitry, digital circuitry, or logic circuitry.

According to the techniques of this disclosure, processing circuitry 264 may receive from at least one of a sensor or sensing circuitry (e.g., electrodes 52 and 60, and/or sensing circuitry 272) a signal indicative of the delivery of a first electrical signal by another medical device (e.g., ICD 9) to an anatomy of patient 14. Processing circuitry 264 may, based on receiving the signal indicative of the delivery of the first electrical signal, control communication circuitry 268 to initiate communication with the another medical device. For example, to initiate communication with the another medical device, processing circuitry 264 may control communication circuitry 268 to communicate with the another medical device, such as such as transmit one or more pairing advertisements, such as Bluetooth pairing advertisements or poll a communication channel.

In some examples, processing circuitry 264 may receive an instruction from the another device via communication circuitry 268 and may respond to the instruction. For example, the instruction may include an instruction to begin delivering therapy (e.g., ATP therapy, pacing therapy, shock(s), etc.), to stop delivering therapy, and/or to configure an operation of IPD 16, such as to reprogram the IPD 16.

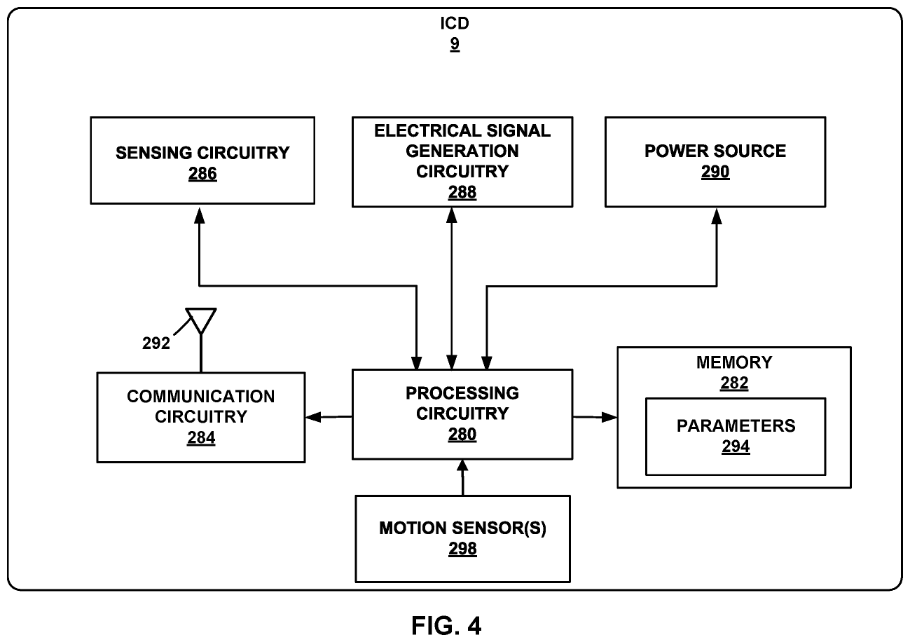

FIG. 4 is a functional block diagram illustrating an example configuration of ICD 9 of the implantable medical device system of FIGS. 1A-1C. In the illustrated example, ICD 9 includes processing circuitry 280, sensing circuitry 286, electrical signal generation circuitry 288, communication circuitry 284, motion sensor(s) 298 (which may include one or more accelerometers), and memory 282. The electronic components may receive power from a power source 290, which may be a rechargeable or non-rechargeable battery. In other examples, ICD 9 may include more or fewer electronic components. The described circuitry may be implemented together on a common hardware component or separately as discrete but interoperable hardware or software components. Depiction of different features as circuitry is intended to highlight different functional aspects and does not necessarily imply that such circuitry must be realized by separate hardware or software components. Rather, functionality associated with one or more circuitry may be performed by separate hardware or software components, or integrated within common or separate hardware or software components. FIG. 4 will be described in the context of ICD 9 being coupled to lead 10 for exemplary purposes only. However, ICD 9 may be coupled to other leads, and thus other electrodes.

Memory 282 includes computer-readable instructions that, when executed by processing circuitry 280, cause ICD 9 and processing circuitry 280 to perform various functions attributed to ICD 9 and processing circuitry 280 herein (e.g., detecting a cardiac event, delivering cardiac therapy, such as anti-tachycardia pacing, and/or generating/transmitting an indication regarding the cardiac event). Memory 282 may include any volatile, non-volatile, magnetic, optical, or electrical media, such as RAM, ROM, FRAM, NVRAM, EEPROM, flash memory, or any other digital or analog media.

Sensing circuitry 286 is electrically coupled to some or all of electrode 28 (or separately to segments 28a and/or 28b) and 32a and 32b via the conductors of lead 10 (shown in FIGS. 1A-1C) and one or more electrical feedthroughs, or to the housing electrode via conductors internal to the housing of ICD 9. Sensing circuitry 286 is configured to obtain signals sensed via one or more combinations of electrode 28 (or separately to segments 28a and/or 28b), 32a, 32b and the housing electrode of ICD 9 and process the obtained signals.

The components of sensing circuitry 286 may be analog components, digital components or a combination thereof. Sensing circuitry 286 may, for example, include one or more sense amplifiers, filters, rectifiers, threshold detectors, ADCs, or the like. Sensing circuitry 286 may convert the sensed signals to digital form and provide the digital signals to processing circuitry 280 for processing or analysis. For example, sensing circuitry 286 may amplify signals from the sensing electrodes and convert the amplified signals to multi-bit digital signals by an ADC. Sensing circuitry 286 may also compare processed signals to a threshold to detect the existence of fiducial points, QRS complexes, and atrial or ventricular depolarizations (e.g., P- or R-waves) and indicate the existence of the atrial depolarization (e.g., P-waves) or ventricular depolarizations (e.g., R-waves) to processing circuitry 280.

Processing circuitry 280 may include any one or more of a microprocessor, a controller, a DSP, an ASIC, an FPGA, or equivalent discrete or analog logic circuitry. In some examples, processing circuitry 280 may include multiple components, such as any combination of one or more microprocessors, one or more controllers, one or more DSPs, one or more ASICs, or one or more FPGAs, as well as other discrete or integrated logic circuitry. The functions attributed to processing circuitry 280 herein may be embodied as software, firmware, hardware or any combination thereof.

Processing circuitry 280 may process the signals from sensing circuitry 286 to monitor electrical activity of the heart of the patient. Processing circuitry 280 may store signals obtained by sensing circuitry 286 as well as any generated ECG waveforms, marker channel data, accelerometer data, or other data derived based on the sensed signals in memory 282. Processing circuitry 280 may analyze the ECG waveforms and/or marker channel data to detect cardiac events (e.g., tachycardia). In response to detecting the cardiac event, processing circuitry 280 may control electrical signal generation circuitry 288 to deliver the desired therapy to treat the cardiac event, e.g., ATP therapy and/or antitachyarrhythmia (e.g., defibrillation or cardioversion) shocks.

Electrical signal generation circuitry 288 is configured to generate and deliver electrical therapy to the heart. Electrical signal generation circuitry 288 may include one or more pulse generators, capacitors, and/or other components capable of generating and/or storing energy to deliver as pacing therapy, defibrillation therapy, cardioversion therapy, cardiac resynchronization therapy, other therapy or a combination of therapies. In some instances, electrical signal generation circuitry 288 may include a first set of components configured to provide pacing therapy and a second set of components configured to provide defibrillation therapy. In other instances, electrical signal generation circuitry 288 may utilize the same set of components to provide both pacing and defibrillation therapy. In still other instances, electrical signal generation circuitry 288 may share some of the defibrillation and pacing therapy components while using other components solely for defibrillation or pacing. In some examples, electrical signal generation circuitry 288 may deliver pacing stimulation, e.g., ATP therapy, in the form of voltage or current electrical pulses. In other examples, electrical signal generation circuitry 288 may deliver stimulation in the form of other signals, such as sine waves, square waves, or other substantially continuous time signals.

Electrical signal generation circuitry 288 is also configured to deliver a first electrical signal, such as a pacing signal, that is used to inform another device, such as IPD 16, to initiate communication with ICD 9. This first electrical signal may be delivered, for example, to heart 26, or other anatomy, which another device may be monitoring. For example, rather than send a wake-up signal via communication circuitry 284 to initiate communication with IPD 16, ICD 9 may send the first electrical signal via electrical signal generation circuitry 288 to an anatomy of patient 14. In some examples, the first electrical signal includes a sub-threshold signal. In some examples, the first electrical signal includes a double pulse (e.g., two pacing pulses). In some examples, the first electrical signal is a signal that is not used for purposes other than indicating to another device that the other device should initiate communication with ICD 9. In this manner, IPD 16 does not need to monitor for a wake-up signal on a communication channel, thereby saving power. Instead, IPD 16 utilizes other sensors or sensing circuitry that is already powered to monitor for physiological signals.

Motion sensor(s) 298 may include a three-axis accelerometer, one or more single-axis accelerometers, or the like. Motion sensor(s) 298 may be configured to generate one or more signals that may be indicative of a posture or activity of patient 14.

Processing circuitry 280 may control electrical signal generation circuitry 288 to deliver the generated therapy or the first electrical signal to the heart via one or more combinations of electrode 28 (or separately to segments 28a and/or 28b), 32a, and 32b of lead 10 and the housing electrode of ICD 9 according to one or more therapy programs, which may be stored in memory 282. In instances in which processing circuitry 280 is coupled to a different lead, other electrodes may be utilized. Processing circuitry 280 controls electrical signal generation circuitry 288 to generate electrical stimulation therapy with the amplitudes, pulse widths, timing, frequencies, electrode combinations or electrode configurations specified by stored therapy programs.

Processing circuitry 280 controls electrical signal generation circuitry 288 to deliver cardiac pacing therapy or the first electrical signal to heart 26 according to parameters, which may be stored in parameters 294 in memory 282. For example, processing circuitry 280 may control electrical signal generation circuitry 288 to deliver pacing pulses with the amplitudes, pulse widths, frequency, or electrode polarities specified by the therapy parameters, including intervals that define under what conditions and when pacing pulses should be delivered.

Processing circuitry 280 may control electrical signal generation circuitry 288 to deliver therapy, such as ATP therapy, antitachyarrhythmia shock, such as defibrillation or cardioversion, or the like. Processing circuitry 280 may also control electrical signal generation circuitry 288 to deliver a first electrical signal to an anatomy of patient 14 according to other parameters which may be stored in parameters 294. The first electrical signal may be a signal that electrical signal generation circuitry 288 may only deliver when processing circuitry 280 is attempting to initiate communications with IPD 16.

Electrical signal generation circuitry 288 may include switch circuitry to select which of the available electrodes are used to deliver the therapy. The switch circuitry may include a switch array, switch matrix, multiplexer, or any other type of switching device suitable to selectively couple electrodes to electrical signal generation circuitry 288. Processing circuitry 280 may select the electrodes to function as signal electrodes, or the signal vector, via the switch circuitry within electrical signal generation circuitry 288. In instances in which defibrillation segments 28a and 28b are each coupled to separate conductors, processing circuitry 280 may be configured to selectively couples electrical signal generation circuitry 288 to either one of segments 28a and 28b individually or couple to both of the segments 28a and 28b concurrently. In some instances, the same switch circuitry may be used by both electrical signal generation circuitry 288 and sensing circuitry 286. In other instances, each of sensing circuitry 286 and electrical signal generation circuitry 288 may have separate switch circuitry.

According to the techniques of this disclosure, processing circuitry 280 may control the electrical signal generation circuitry to deliver a first electrical signal to an anatomy of the patient. Processing circuitry 280 may, based on the electrical signal generation circuitry delivering the first electrical signal, communicate with another medical device via the communication circuitry.

In one example, electrical signal generation circuitry 288 may deliver pacing via an electrode vector that includes one or both defibrillation electrode segments 28a and 28b. The electrode vector used for pacing may be segment 28a as an anode (or cathode) and one of segment 28b, or electrodes 32a or 32b, or the housing of ICD 9 as the cathode (or anode) or segment 28b as an anode (or cathode) and one of segment 28b, or electrodes 32a or 32b, or the housing of ICD 9 as the cathode (or anode). In some examples, electrode 52 and/or electrode 60 of IPD 16 may be used as an anode or cathode and ICD 9 may communicate with IPD 16 via communication circuitry 284 and/or external device may communicate with ICD 9 and IPD 16 to coordinate the use of electrodes of both ICD 9 and IPD 16.

Processing circuitry 280 controls electrical signal generation circuitry 288 to generate and deliver pacing pulses with any of a number of shapes, amplitudes, pulse widths, or other characteristic to capture the heart. For example, the pacing pulses may be monophasic, biphasic, or multi-phasic (e.g., more than two phases). The pacing thresholds of the heart when delivering pacing pulses from the substernal space, e.g., from electrodes 32a, 32b and/or electrode segments 28a and/or 28b substantially within anterior mediastinum 36, may depend upon a number of factors, including location, type, size, orientation, and/or spacing of electrodes 32a and 32b and/or electrode segments 28a and 28b, location of ICD 9 relative to electrodes 32a and 32b and/or electrode segments 28a and 28b, physical abnormalities of the heart (e.g., pericardial adhesions or myocardial infarctions), or other factor(s).

Processing circuitry 280 may include a timing and control circuitry, which may be embodied as hardware, firmware, software, or any combination thereof. The timing and control circuitry may comprise a dedicated hardware circuit, such as an ASIC, separate from other processing circuitry 280 components, such as a microprocessor, or a software module executed by a component of processing circuitry 280, which may be a microprocessor or ASIC. The timing and control circuitry may implement programmable counters. If ICD 9 is configured to generate and deliver pacing pulses to heart 26, such counters may control the basic time intervals associated with DDD, VVI, DVI, VDD, AAI, DDI, DDDR, VVIR, DVIR, VDDR, AAIR, DDIR and other modes of pacing.

Intervals defined by the timing and control circuitry within processing circuitry 280 may include atrial and ventricular pacing escape intervals, refractory periods during which sensed P-waves and R-waves are ineffective to restart timing of the escape intervals, and the pulse widths of the pacing pulses. As another example, the timing and control circuitry may withhold sensing from one or more channels of sensing circuitry 286 for a time interval during and after delivery of electrical stimulation to heart 26. The durations of these intervals may be determined by processing circuitry 264 in response to stored data in memory 282. The timing and control circuitry of processing circuitry 264 may also determine the amplitude of the cardiac pacing pulses.

Interval counters implemented by the timing and control circuitry of processing circuitry 280 may be reset upon sensing of R-waves and P-waves with detection channels of sensing circuitry 286. In examples in which ICD 9 provides pacing, electrical signal generation circuitry 288 may include pacer output circuits that are coupled to electrodes, for example, appropriate for delivery of a bipolar or unipolar pacing pulse to one of the chambers of heart 26. In such examples, processing circuitry 280 may reset the interval counters upon the generation of pacing pulses by electrical signal generation circuitry 288, and thereby control the basic timing of cardiac pacing functions, including ATP or post-shock pacing.

The value of the count present in the interval counters when reset by sensed R-waves and P-waves may be used by processing circuitry 280 to measure the durations of R-R intervals, P-P intervals, P-R intervals and R-P intervals, which are measurements that may be stored in memory 282. Processing circuitry 280 may use the count in the interval counters to detect a tachyarrhythmia event, such as atrial fibrillation, atrial tachycardia, ventricular fibrillation, or ventricular tachycardia. These intervals may also be used to detect the overall heart rate, ventricular contraction rate, and heart rate variability. A portion of memory 282 may be configured as a plurality of recirculating buffers, capable of holding series of measured intervals, which may be analyzed by processing circuitry 280 in response to the occurrence of a pace or sense interrupt to determine whether the patient's heart 26 is presently exhibiting atrial or ventricular tachyarrhythmia.

In some examples, an arrhythmia detection method may include any suitable tachyarrhythmia detection algorithms. In some examples, processing circuitry 280 may determine that tachyarrhythmia has occurred by identification of shortened R-R (or P-P) interval lengths. Generally, processing circuitry 280 detects tachycardia when the interval length falls below 220 milliseconds and fibrillation when the interval length falls below 180 milliseconds. In other examples, processing circuitry 280 may detect ventricular tachycardia when the interval length falls between 330 milliseconds and ventricular fibrillation when the interval length falls below 240 milliseconds. These interval lengths are merely examples, and a user may define the interval lengths as desired, which may then be stored within memory 282. This interval length may need to be detected for a certain number of consecutive cycles, for a certain percentage of cycles within a running window, or a running average for a certain number of cardiac cycles, as examples. In other examples, additional physiological parameters may be used to detect an arrhythmia. For example, processing circuitry 280 may analyze one or more morphology measurements, impedances, or any other physiological measurements to determine that patient 14 is experiencing a tachyarrhythmia.

In the event that an ATP regimen is desired, timing intervals for controlling the generation of ATP therapies by therapy deliver circuitry 288 may be loaded by processing circuitry 280 into the timing and control circuitry based on ATP parameters in parameters 294 to control the operation of the escape interval counters therein and to define refractory periods during which detection of R-waves and P-waves is ineffective to restart the escape interval counters for the ATP. An ATP regimen may be desired if processing circuitry 280 detects an atrial or ventricular tachyarrhythmia based on signals from sensing circuitry 286, and/or receives a command from another device or system, such as IPD 16, as examples.

Memory 282 may be configured to store a variety of operational parameters, therapy parameters, including ATP therapy parameters, sensed and detected data, and any other information related to the therapy and treatment of patient 14. In the example of FIG. 4, memory 282 may store sensed ECGs, detected arrhythmias, communications from IPD 16, and therapy parameters that define ATP therapy (which may be stored in parameters 294). In other examples, memory 282 may act as a temporary buffer for storing data until it can be uploaded to IPD 16, another implanted device, or external device 21.

Communication circuitry 284 includes any suitable hardware, firmware, software or any combination thereof for communicating with another device, such as external device 21 (FIGS. 1A-1C and 7), IPD 16 (FIGS. 1A-1C and FIG. 2), a clinician programmer, a patient monitoring device, or the like. For example, communication circuitry 284 may include appropriate modulation, demodulation, frequency conversion, filtering, and amplifier components for transmission and reception of data with the aid of antenna 292. Antenna 292 may be located within connector block of ICD 9 or within the housing of ICD 9. Under the control of processing circuitry 280, communication circuitry 284 may receive downlink telemetry from and send uplink telemetry to external device 21 and/or IPD 16 with the aid of antenna 292, which may be internal and/or external. Processing circuitry 280 may provide the data to be uplinked to external device 21 and/or IPD 16 and the control signals for the telemetry circuit within communication circuitry 284, e.g., via an address/data bus. In some examples, communication circuitry 284 may provide received data to processing circuitry 280 via a multiplexer.

In some examples, ICD 9 may signal external device 21 to further communicate with and pass the alert through a network such as the Medtronic CareLink® Network developed by Medtronic plc, of Dublin, Ireland, or some other network linking patient 14 to a clinician. ICD 9 may spontaneously transmit information to the network or in response to an interrogation request from a user.

Power source 290 may be any type of device that is configured to hold a charge to operate the circuitry of ICD 9. Power source 290 may be provided as a rechargeable or non-rechargeable battery. In other example, power source 290 may incorporate an energy scavenging system that stores electrical energy from movement of ICD 9 within patient 14.

The various circuitry of ICD 9 may include any one or more processors, controllers, DSPs, ASICs, FPGAs, or equivalent discrete or integrated circuitry, including analog circuitry, digital circuitry, or logic circuitry.

FIG. 5 is a functional block diagram illustrating an example configuration of external device 21 of FIGS. 1A-1C. External device 21 may include processing circuitry 400, memory 402, communication circuitry 408, user interface 406, motion sensor(s) 410 (which may include one or more accelerometers), and power source 404. Processing circuitry 400 controls user interface 406 and communication circuitry 408, and stores and retrieves information and instructions to and from memory 402. External device 21 may be configured for use as a clinician programmer or a patient programmer. Processing circuitry 400 may comprise any combination of one or more processors including one or more microprocessors, DSPs, ASICs, FPGAs, or other equivalent integrated or discrete logic circuitry. Accordingly, processing circuitry 400 may include any suitable structure, whether in hardware, software, firmware, or any combination thereof, to perform the functions ascribed herein to processing circuitry 400.

In some examples, motion sensor(s) 410 may include a three-axis accelerometer, one or more single-axis accelerometers, or the like. In examples, where external device 21 may be a smart phone carried by patient 14 (e.g., in a pocket of patient 14), a wearable device, or the like, motion sensor(s) 410 may be configured to generate one or more signals that may be indicative of a posture and/or activity of patient 14.

A user, such as a clinician or patient 14, may interact with external device 21 through user interface 406. User interface 406 may include a display, such as an LCD or LED display or other type of screen, to present information related the ATP therapy, including ATP therapy parameters stored in parameters 412 or in memory of an IMD, or to display a visual indication or provide audio indication relating to a cardiac event to the user. In addition, user interface 406 may include an input mechanism to receive input from the user. The input mechanisms may include, for example, buttons, a keypad (e.g., an alphanumeric keypad), a peripheral pointing device or another input mechanism that allows the user to navigate through user interfaces presented by processing circuitry 400 of external device 21 and provide input.

If external device 21 includes buttons and a keypad, the buttons may be dedicated to performing a certain function, i.e., a power button, or the buttons and the keypad may be soft keys that change in function depending upon the section of the user interface currently viewed by the user. Alternatively, a screen of external device 21 may be a touch screen that allows the user to provide input directly to the user interface shown on the display. The user may use a stylus or a finger to provide input to the display. In other examples, user interface 406 also includes audio circuitry for providing audible instructions or sounds, such as an indication regarding a cardiac event, to patient 14 and/or receiving voice commands from patient 14, which may be useful if patient 14 has limited motor functions. Patient 14, a clinician or another user may also interact with external device 21 to manually select therapy programs, generate new therapy programs, modify therapy programs through individual or global adjustments, and transmit the new programs ICD 9 and/or IPD 16.

In some examples, when external device 21 transmits new programs or commands to IPD 16, external device transmits the new programs or commands via ICD 9. Any data that may be retrieved or received by external device 21 from IPD 16 may also be via IPD 16. For example, when external device 21 is attempting to transmit a new program to IPD 16, external device 21 may transmit the new program to ICD 9 with an indication that the new program is intended for IPD 16. ICD 9 may then send the first electrical signal to heart 26. IPD 16, which is monitoring the electrical activity of heart 26 may sense the first electrical signal and initiate communication via communication circuitry 268 (FIG. 3) and communication circuitry 284 (FIG. 4). ICD 9 may then transmit the new program over a communication channel to IPD 16. In this manner, IPD 16 does not have to monitor for a communication wake-up signal on the communication channel from external device 21 using communication circuitry 268.

In some examples, at least some of the control of therapy delivery by ICD 9 and/or IPD 16 may be implemented by processing circuitry 400 of external device 21. For example, in some examples, processing circuitry 400 may control delivery of cardiac therapy, such as ATP therapy by ICD 9 and/or IPD 16 by communicating with by ICD 9 and/or IPD 16 and may receive data regarding sensed signals and by communicating with by ICD 9 and/or IPD 16 to control electrical signal generation circuitry of any of by ICD 9 and/or IPD 16. In some examples, memory 402 may store ATP therapy parameters, may use the parameters to control electrical signal generation circuitry of by ICD 9 and/or IPD 16, and/or may modify ATP therapy parameters.

Memory 402 may include instructions for operating user interface 406 and communication circuitry 408, for managing power source 404, and for performing any functions attributed to external device 21 (e.g., determining a cardiac event, controlling the delivery of therapy, and/or generating an indication for output). Memory 402 may also store any therapy data retrieved from PCD 110 during the course of therapy, accelerometer data, or other sensed data. The clinician may use this therapy data to determine the progression of the patient condition in order to predict future treatment. Memory 402 may include any volatile or nonvolatile memory, such as RAM, ROM, FRAM, EEPROM or flash memory. Memory 402 may also include a removable memory portion that may be used to provide memory updates or increases in memory capacities. A removable memory may also allow sensitive patient data to be removed before external device 21 is used by a different patient.

Wireless telemetry in external device 21 may be accomplished by RF communication or proximal inductive interaction of external device 21, ICD 9, and/or IPD 16. This wireless communication is possible through the use of communication circuitry 408 with the aid of antenna 414, which may communicate with a proprietary protocol or industry-standard protocol such as using the Bluetooth specification set. Accordingly, communication circuitry 408 may be similar to the communication circuitry contained within by ICD 9 and/or IPD 16. In alternative examples, external device 21 may be capable of infrared communication or direct communication through a wired connection. In this manner, other external devices may be capable of communicating with external device 21 without needing to establish a secure wireless connection.

Power source 404 may deliver operating power to the components of external device 21. Power source 404 may include a battery and a power generation circuit to produce the operating power. In some examples, the battery may be rechargeable to allow extended operation. Recharging may be accomplished by electrically coupling power source 404 to a cradle or plug that is connected to an AC outlet. In addition, recharging may be accomplished through proximal inductive interaction between an external charger and an inductive charging coil within external device 21. In other examples, traditional batteries (e.g., nickel cadmium or lithium ion batteries) may be used. In addition, external device 21 may be directly coupled to an alternating current outlet to operate. Power source 404 may include circuitry to monitor power remaining within a battery. In this manner, user interface 406 may provide a current battery level indicator or low battery level indicator when the battery needs to be replaced or recharged. In some cases, power source 404 may be capable of estimating the remaining time of operation using the current battery.

FIG. 6 is a flowchart illustrating example communication techniques for a first medical device in accordance with one or more aspects of this disclosure. The discussion of FIG. 6 is in the context of ICD 9 of FIG. 4 for ease of explanation, however these techniques may be performed by any of the devices described herein which are capable of performing such techniques.

Processing circuitry 280 may control electrical signal generation circuitry 288 to deliver a first electrical signal to an anatomy of patient 14 (600). For example, processing circuitry 280 may control electrical signal generation circuitry 288 to deliver one or more pacing pulses to heart 26. In some examples, the one or more pacing pulses include at least one of subthreshold energy pulse or a double pulse. Such a first electrical signal may be a signal that is only used when attempting to initiate communication with another medical device or between other medical devices. For example, the first electrical signal may represent non-channel based signaling to request communication handshaking with another medical device, such as IPD 16.

Processing circuitry 280 may control, based on electrical signal generation circuitry 288 delivering the first electrical signal, communication circuitry 284 to communicate with another medical device (602). For example, the another medical device (e.g., IPD 16) may send a communication, such as transmit one or more pairing advertisements, such as Bluetooth pairing advertisements or begin polling a communication channel in response to sensing the first electrical signal, for example, when monitoring anatomy, such as heart 26. Such advertisements or polling may initiate communication between processing circuitry 280 and the another medical device.

In some examples, processing circuitry 280 may refrain from controlling the communication circuitry to send a wake-up signal to the another medical device. For example, processing circuitry 280 may use the first electrical signal in lieu of sending a communication wake-up signal over a communication channel to the another medical device. In some examples, as part of communicating with the another medical device, communication circuitry 284 is configured to receive a communication from the another medical device. In some examples, as part of communicating with the another medical device, communication circuitry 284 is configured to send a communication to the another medical device.

In some examples, processing circuitry 280 may determine to control electrical signal generation circuitry 288 to deliver the first electrical signal based on at least one sensed physiological parameter (e.g., by sensing circuitry 286) or a communication from external device 21. In some examples, the at least one physiological parameter is indicative of a cardiac event, such as tachycardia.

In some examples, processing circuitry 280 controls communication circuitry 284 to send an instruction to the another medical device. In some examples, the instruction includes one or more of an instruction to begin delivering therapy (e.g., ATP therapy, pacing pulses, shock(s), etc.), to stop delivering therapy, or to configure an operation of the second IMD, such as to reprogram the another medical device.

FIG. 7 is a flowchart illustrating example communication techniques for a second medical device in accordance with one or more aspects of this disclosure. The discussion of FIG. 7 is in the context of IPD 16 of FIG. 3 for ease of explanation, however these techniques may be performed by any of the devices described herein which are capable of performing such techniques.

Processing circuitry 264 may receive, from at least one of a sensor or sensing circuitry (e.g., electrodes 52, electrode 60 and/or sensing circuitry 272), a signal (700). For example, sensing circuitry 272 and/or electrodes 52 or electrode 60 may sense the signal. Processing circuitry 264 may determine that the signal is indicative of delivery of a first electrical signal from another medical device (e.g., ICD 9) to an anatomy of patient 14 (702). For example, processing circuitry 264 may compare the sensed signal to parameters stored in parameters 276 that are indicative of the delivery of the first electrical signal to anatomy (e.g., heart 26) of patient 14 to determine that the signal is indicative of the delivery of the first electrical signal.

Processing circuitry 264 may control, based on receiving the signal indicative of the delivery of the first electrical signal, communication circuitry 268 to initiate communication with at least one of the another medical device or a third medical device (704). For example, processing circuitry 264 may control communication circuitry 268 to send a communication to at least one of the another medical device or the third medical device, such as transmit one or more pairing advertisements, such as Bluetooth pairing advertisements. Additionally, or alternatively, processing circuitry 264 may control communication circuitry 268 to poll a communication channel.

In some examples, processing circuitry 264 may receive, via communication circuitry 268, an instruction from the another device. For example, communication circuitry 268 may receive an instruction from ICD 9 and may send the instruction to processing circuitry 264. In some examples, the instruction includes an instruction to control electrical signal generation circuitry to deliver therapy based on the received instruction. In some examples, the instruction includes an instruction to configure an operation of the IPD 16.

For example, a clinician may enter a new program or different therapy parameters into user interface 406 of external device 21 (FIG. 5). External device may send the new program or different therapy parameters to ICD 9 which may be monitoring for a communication wake up signal. ICD 9 may send the first electrical signal to heart 26, IPD 16 may initiate communication with ICD 9, and ICD 9 may send the instruction to IPD 16.

Processing circuitry 264 may respond to the instruction. For example, processing circuitry 264 may control electrical signal generation circuitry 270 to deliver therapy based on the received instruction or may configure an operation of the medical device. In some examples, the therapy includes anti-tachyarrhythmia pacing therapy.

In some examples, the first electrical signal comprises one or more pacing pulses. In some examples, the one or more pacing pulses comprise at least one of a subthreshold energy pulse or a double pulse.

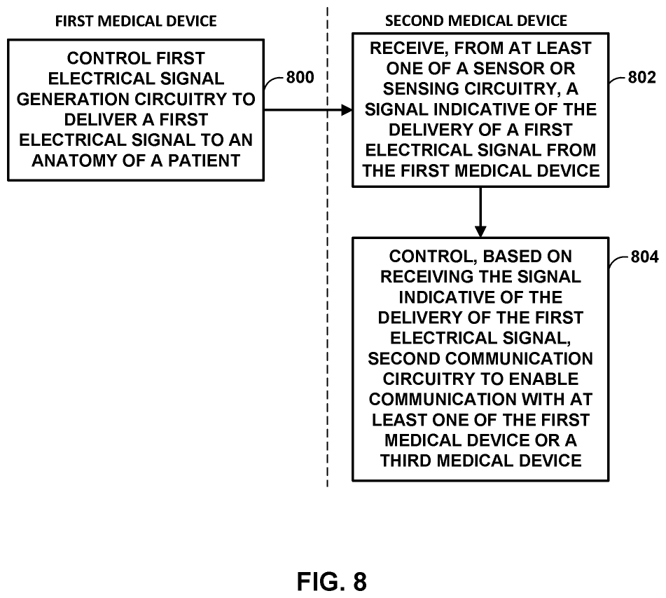

FIG. 8 is a flowchart illustrating other example communication techniques for a first medical device and a second medical device in accordance with one or more aspects of this disclosure. The discussion of FIG. 8 is in the context of system 8 of FIGS. 1A-1C, including ICD 9 of FIG. 5, and IPD 16 of FIGS. 2-3 for ease of explanation, however these techniques may be performed by any of the devices described herein which are capable of performing such techniques.

Processing circuitry 280 of ICD 9 may control electrical signal generation circuitry 288 to deliver a first electrical signal to an anatomy of patient 14 (800). For example, processing circuitry 280 may control electrical signal generation circuitry 288 to deliver an electrical signal to heart 26 of patient 14 that processing circuitry 280 may only use to initiate communication with another medical device (e.g., IPD 16) or between other medical devices.

Processing circuitry 264 of IPD 16 may receive from at least one of a sensor or sensing circuitry (e.g., electrodes 52 and/or 60 and/or sensing circuitry), a signal indicative of the delivery of the first electrical signal by the first medical device (e.g., ICD 9) to the anatomy (e.g., heart 26) of patient 14 (802). For example, sensing circuitry 272 and/or electrodes 52 and/or 60 may monitor heart 26 for electrical signals, may sense the first electrical signal and may send the first electrical signal to processing circuitry 264.

Processing circuitry 264 may control, based on receiving the signal indicative of the delivery of the first electrical signal, communication circuitry 268 to initiate communication with at least one of the first medical device (e.g., ICD 9) or a third medical device (804). For example, IPD 16 may send a communication to ICD 9 and/or the third medical device, such as transmit one or more pairing advertisements, such as Bluetooth pairing advertisements, or IPD 16 may begin polling a communication channel in response to receiving the signal indicative of the delivery of the first electrical signal. For example, IPD 16 may be configured to recognize the first electrical signal as being a trigger for IPD 16 to initiate communication with ICD 9 and/or the third medical device.

In some examples, controlling communication circuitry 268 to initiate communication with at least one of the first medical device (ICD 9) or a third medical device includes sending a communication to the first medical device. In some examples, processing circuitry 280 may receive, based on electrical signal generation circuitry 288 delivering the first electrical signal, the communication from the second medical device (IPD 16) via communication circuitry 284. In some examples, processing circuitry 280 may control, based on receiving the communication from the second medical device, communication circuitry 284 to send an instruction to the second medical device. In some examples, processing circuitry 264 may receive, via communication circuitry 268, the instruction from the first medical device (ICD 9) and processing circuitry 264 may respond to the instruction.

Any suitable modifications may be made to the techniques described herein and any suitable device, processing circuitry, electrical signal generation circuitry, and/or electrodes may be used for performing the steps of the methods described herein. The steps of the methods may be performed by any suitable number of devices. For example, a processing circuitry of one device may perform some of the steps while a electrical signal generation circuitry and/or sensing circuitry of another device may perform other steps of the method, while communication circuitry may allow for communication needed for the processing circuitry to receive information from other devices. This coordination may be performed in any suitable manner according to particular needs.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, FRAM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

The techniques described in this disclosure, including those attributed to ICD 9, IPD 16, PCD 110, ICM 300, and external device 21, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, remote servers, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. For example, any of the techniques or processes described herein may be performed within one device or at least partially distributed amongst two or more devices, such as between ICD 9, IPD 16, PCD 110, ICM 300, and/or external device 21. In addition, any of the described units, circuitry or components may be implemented together or separately as discrete but interoperable logic devices. Depiction of different features as circuitry is intended to highlight different functional aspects and does not necessarily imply that such circuitry must be realized by separate hardware or software components. Rather, functionality associated with one or more circuitry may be performed by separate hardware or software components, or integrated within common or separate hardware or software components.

The techniques described in this disclosure may also be embodied or encoded in an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media.

As used herein, the term “circuitry” refers to an application specific integrated circuit (ASIC), an electronic circuit, a processor (shared, dedicated, or group) and memory that execute one or more software or firmware programs, a combinational logic circuit, or other suitable components that provide the described functionality.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

Various examples have been described for delivering cardiac stimulation therapies as well as coordinating the operation of various devices within a patient. Any combination of the described operations or functions is contemplated. These and other examples are within the scope of the following claims.

The following examples are a non-limiting list of clauses in accordance with one or more techniques of this disclosure.

Example 1. A medical device comprising: memory configured to store parameters for a first electrical signal for a patient; communication circuitry; electrical signal generation circuitry; and processing circuitry communicatively coupled to the memory, the communication circuitry, and the electrical signal generation circuitry, the processing circuitry being configured to: control the electrical signal generation circuitry to deliver the first electrical signal to an anatomy of the patient; and based on the electrical signal generation circuitry delivering the first electrical signal, control the communication circuitry to communicate with another medical device.

Example 2. The medical device of Example 1, wherein the processing circuitry is further configured to refrain from controlling the communication circuitry to send a wake-up signal to the another medical device.

Example 3. The medical device of Example 1 or Example 2, wherein as part of communicating with the another medical device, the communication circuitry is configured to receive a communication from the another medical device.

Example 4. The medical device of any of Examples 1-3, wherein as part of communicating with the another medical device, the communication circuitry is configured to send a communication to the another medical device.

Example 5. The medical device of any of Examples 1-4, further comprising at least one of a sensor or sensing circuitry configured to sense physiological parameters of the patient, wherein the processing circuitry is further configured to determine to control the electrical signal generation circuitry to deliver the first electrical signal based on at least one sensed physiological parameter or a communication from an external device.

Example 6. The medical device of Example 5, wherein the at least one sensed physiological parameter is indicative of a cardiac event.

Example 7. The medical device of Example 6, wherein the cardiac event comprises tachycardia.

Example 8. The medical device of any of Examples 1-7, wherein the processing circuitry is further configured to control the communication circuitry to send an instruction to the another medical device.

Example 9. The medical device of Example 8, wherein the instruction comprises one or more of: an instruction to begin delivering pacing pulses to a heart of a patient; an instruction to stop delivering pacing pulses to the heart of the patient; or an instruction comprising configuration information for configuring an operation of the another medical device.

Example 10. The medical device of any of Examples 1-9, wherein the first electrical signal comprises one or more pacing pulses.

Example 11. The medical device of Example 10, wherein the one or more pacing pulses comprise at least one of a subthreshold energy pulse or a double pulse.

Example 12. The medical device of any of Examples 1-11, wherein the medical device comprises an extracardiovascular defibrillator.

Example 13. A medical device comprising: memory configured to store parameters indicative of delivery of a first electrical signal to an anatomy of a patient;

communication circuitry; at least one of a sensor or sensing circuitry; and processing circuitry communicatively coupled to the memory, the communication circuitry, and the at least one of the sensor or the sensing circuitry, the processing circuitry being configured to: receive from the at least one of the sensor or the sensing circuitry a signal; determine that the signal is indicative of the delivery of the first electrical signal by another medical device to the anatomy of the patient; and based on receiving the signal indicative of the delivery of the first electrical signal, control the communication circuitry to initiate communication with at least one of the another medical device or a third medical device.

Example 14. The medical device of Example 13, wherein as part of controlling the communication circuitry to initiate communication with the another medical device, the processing circuitry is configured to control the communication circuitry to send a communication to at least one of the another medical device or the third medical device.

Example 15. The medical device of Example 13 or Example 14, wherein as part of controlling the communication circuitry to initiate communication with the another medical device, the processing circuitry is configured to control the communication circuitry to poll a communication channel.

Example 16. The medical device of any of Examples 13-15, wherein the processing circuitry is further configured to: receive an instruction from the another medical device via the communication circuitry; and respond to the instruction.

Example 17. The medical device of Example 16, further comprising electrical signal generation circuitry and wherein as part of responding to the instruction, the processing circuitry is configured to control the electrical signal generation circuitry to deliver therapy based on the received instruction.

Example 18. The medical device of Example 17, wherein the therapy comprises anti-tachyarrhythmia pacing therapy.

Example 19. The medical device of any of Examples 16-18, wherein as part of responding to the instruction, the processing circuitry is configured to configure an operation of the medical device based on the received instruction.

Example 20. The medical device of any of Examples 13-19, wherein the medical device comprises a pacemaker.

Example 21. The medical device of any of Examples 13-20, wherein the first electrical signal comprises one or more pacing pulses.

Example 22. The medical device of Example 21, wherein the one or more pacing pulses comprise at least one of a subthreshold energy pulse or a double pulse.

Example 23. A method comprising: controlling, by processing circuitry, electrical signal generation circuitry to deliver a first electrical signal to an anatomy of a patient; and controlling, by the processing circuitry and based on the electrical signal generation circuitry delivering the first electrical signal, communication circuitry to communicate with another medical device.

Example 24. The method of Example 23, further comprising refraining, by the processing circuitry, from controlling the communication circuitry to send a wake-up signal to the another medical device.

Example 25. The method of Example 23 or Example 24, wherein communicating with the another medical device comprises receiving a communication from the another medical device.

Example 26. The method of any of Examples 23-25, wherein communicating with the another medical device comprises sending a communication to the another medical device.

Example 27. The method of any of Examples 23-26, further comprising determining to control the electrical signal generation circuitry to deliver the first electrical signal based on at least one sensed physiological parameter or a communication from an external device.

Example 28. The method of Example 27, wherein the one or more sensor signals are indicative of a cardiac event.

Example 29. The method of Example 28, wherein the cardiac event comprises tachycardia.

Example 30. The method of any of Examples 23-29, further comprising controlling, by the processing circuitry, the communication circuitry to send an instruction to the another medical device.

Example 31. The method of any of Examples 23-30, wherein the instruction comprises one or more of: an instruction to deliver pacing pulses to a heart of a patient; an instruction to stop delivering pacing pulses to the heart of the patient; or configuration information for configuring an operation of the another medical device.

Example 32. The method of any of Examples 23-31, wherein the first electrical signal comprises one or more pacing pulses.

Example 33. The method of Example 32, wherein the one or more pacing pulses comprise at least one of a subthreshold energy pulse or a double pulse.

Example 34. A method comprising: receiving, by processing circuitry of a medical device and from at least one of a sensor or sensing circuitry, a signal; determining that the signal is indicative of the delivery of a first electrical signal by another medical device to an anatomy of a patient; and controlling, by the processing circuitry and based on receiving the signal indicative of delivery of the first electrical signal, communication circuitry to initiate communication with at least one of the another medical device or a third medical device.

Example 35. The method of Example 34, wherein controlling the communication circuitry to initiate communication with the another medical device comprises controlling the communication circuitry to send a communication to at least one of the another medical device or the third medical device.

Example 36. The method of any of Example 34 or Example 35, wherein controlling the communication circuitry to initiate communication with the another medical device comprises controlling the communication circuitry to poll a communication channel.

Example 37. The method of any of Examples 34-36, further comprising: receiving, by the processing circuitry and via the communication circuitry, an instruction from the another medical device; and responding, by the processing circuitry, to the instruction.

Example 38. The method of Example 37, wherein responding to the instruction comprises an instruction to control electrical signal generation circuitry to deliver therapy based on the received instruction.

Example 39. The method of Example 38, wherein the therapy comprises anti-tachyarrhythmia pacing therapy.

Example 40. The method of any of Examples 34-39, wherein responding to the instruction comprises an instruction to configure an operation of the medical device.

Example 41. The method of any of Examples 34-40, wherein the first electrical signal comprises one or more pacing pulses.

Example 42. The method of Example 41, wherein the one or more pacing pulses comprise at least one of a subthreshold energy pulse or a double pulse.

Example 43. A method comprising: controlling, by first processing circuitry of a first medical device, electrical signal generation circuitry to deliver a first electrical signal to an anatomy of a patient; receiving, by second processing circuitry of a second medical device and from at least one of a sensor or sensing circuitry, a signal indicative of delivery of the first electrical signal from the first medical device to the anatomy of the patient; controlling, by the second processing circuitry and based on receiving the signal indicative of the delivery of the first electrical signal, second communication circuitry to initiate communication with at least one of the first medical device or a third medical device.

Example 44. The method of Example 43, wherein controlling the second communication circuitry to initiate communication with at least one of the first medical device or a third medical device comprises sending a communication to the first medical device, the method further comprising: receiving, by the first processing circuitry and based on the electrical signal generation circuitry delivering the first electrical signal, the communication from the second medical device via first communication circuitry; controlling, by the first processing circuitry and based on receiving the communication from the second medical device, the first communication circuitry to send an instruction to the second medical device; receiving, by the second processing circuitry and via the second communication circuitry, the instruction from the first medical device; and responding, by the second processing circuitry, to the instruction.

Example 45. A non-transitory computer-readable storage medium having stored thereon instructions that, when executed, cause processing circuitry to: control electrical signal generation circuitry to deliver a first electrical signal; and based on the electrical signal generation circuitry delivering the first electrical signal, communicate with another medical device via communication circuitry.

Example 46. A non-transitory computer-readable storage medium having stored thereon instructions that, when executed, cause processing circuitry to: receive from at least one of a sensor or sensing circuitry a signal; determine that the signal is indicative of delivery of a first electrical signal from another medical device to anatomy of a patient; and based on receiving the signal indicative of the delivery of the first electrical signal, control communication circuitry to initiate communication with the another medical device.

1. A medical device comprising:
memory configured to store parameters for a first electrical signal for a patient;
communication circuitry;
electrical signal generation circuitry; and
processing circuitry communicatively coupled to the memory, the communication circuitry, and the electrical signal generation circuitry, the processing circuitry being configured to:
control the electrical signal generation circuitry to deliver the first electrical signal to an anatomy of the patient; and
based on the electrical signal generation circuitry delivering the first electrical signal, control the communication circuitry to communicate with another medical device.

2. The medical device of claim 1, wherein the processing circuitry is further configured to refrain from controlling the communication circuitry to send a wake-up signal to the another medical device.

3. The medical device of claim 1, wherein as part of communicating with the another medical device, the communication circuitry is configured to one or more of receive a communication from the another medical device or send a communication to the another medical device.

4. The medical device of claim 1, further comprising at least one of a sensor or sensing circuitry configured to sense physiological parameters of the patient, wherein the processing circuitry is further configured to determine to control the electrical signal generation circuitry to deliver the first electrical signal based on at least one sensed physiological parameter or a communication from an external device.

5. The medical device of claim 4, wherein the at least one sensed physiological parameter is indicative of a cardiac event.

6. The medical device of claim 5, wherein the cardiac event comprises tachycardia.

7. The medical device of claim 1, wherein the processing circuitry is further configured to control the communication circuitry to send an instruction to the another medical device.

8. The medical device of claim 7, wherein the instruction comprises one or more of:
an instruction to begin delivering pacing pulses to a heart of a patient;
an instruction to stop delivering pacing pulses to the heart of the patient; or
an instruction comprising configuration information for configuring an operation of the another medical device.

9. The medical device of claim 1, wherein the first electrical signal comprises one or more pacing pulses.

10. The medical device of claim 9, wherein the one or more pacing pulses comprise at least one of a subthreshold energy pulse or a double pulse.

11. The medical device of claim 1, wherein the medical device comprises an extracardiovascular defibrillator.

12. A medical device comprising:
memory configured to store parameters indicative of delivery of a first electrical signal to an anatomy of a patient;
communication circuitry;
at least one of a sensor or sensing circuitry; and
processing circuitry communicatively coupled to the memory, the communication circuitry, and the at least one of the sensor or the sensing circuitry, the processing circuitry being configured to:
receive from the at least one of the sensor or the sensing circuitry a signal;
determine that the signal is indicative of the delivery of the first electrical signal by another medical device to the anatomy of the patient; and
based on receiving the signal indicative of the delivery of the first electrical signal, control the communication circuitry to initiate communication with at least one of the another medical device or a third medical device.

13. The medical device of claim 12, wherein as part of controlling the communication circuitry to initiate communication with the another medical device, the processing circuitry is configured to control the communication circuitry to send one or more of a communication to at least one of the another medical device or the third medical device or control the communication circuitry to poll a communication channel.

14. The medical device of claim 12, wherein the processing circuitry is further configured to:
receive an instruction from the another medical device via the communication circuitry; and
respond to the instruction.

15. The medical device of claim 14, further comprising electrical signal generation circuitry and wherein as part of responding to the instruction, the processing circuitry is configured to control the electrical signal generation circuitry to deliver therapy based on the received instruction.

16. The medical device of claim 15, wherein the therapy comprises anti-tachyarrhythmia pacing therapy.

17. The medical device of claim 14, wherein as part of responding to the instruction, the processing circuitry is configured to configure an operation of the medical device based on the received instruction.

18. The medical device of claim 12, wherein the medical device comprises a pacemaker.

19. The medical device of claim 12, wherein the first electrical signal comprises one or more pacing pulses.

20. A method comprising:
controlling, by processing circuitry, electrical signal generation circuitry to deliver a first electrical signal to an anatomy of a patient; and
controlling, by the processing circuitry and based on the electrical signal generation circuitry delivering the first electrical signal, communication circuitry to communicate with another medical device.
